(19) United States
(12) Patent Application Publication
(10) Pub. No.: US 2026/0308202 A1
(43) Pub. Date: Oct. 8, 2026

(54) USING VEFG165 FRAGMENTS AS A NOVEL BIOMARKER OF INFLAMMATION TO DIAGNOSE AND GUIDE MEDICAL CARE AND TREATMENTS

(71) Applicant: THE CHILDREN'S MEDICAL CENTER CORPORATION, Boston, MA (US)

(72) Inventors: Avner ADINI, Brookline, MA (US); Benjamin D. MATTHEWS, West Newton, MA (US)

(73) Assignee: THE CHILDREN'S MEDICAL CENTER CORPORATION, Boston, MA (US)

(21) Appl. No.: 19/473,934

(22) PCT Filed: Apr. 4, 2024
(86) PCT No.: PCT/US2024/022928

§ 371 Date: Oct. 9, 2025

Related U.S. Application Data
(60) Provisional application No. 63/459,399, filed on Apr. 14, 2023.
(60) Provisional application No. 63/613,857, filed on Dec. 22, 2023.

(51) Int. Cl. C07K 7/08; A61K 38/00; A61P 29/00; G01N 33/74

(52) U.S. Cl. CPC C07K 7/08; A61P 29/00; G01N 33/74; A61K 38/00

(57) ABSTRACT

Described herein are peptides and pharmaceutical compositions, e.g., for the treatment of inflammation. Further described herein are methods of treating patients for inflammation and/or infection with particularly efficacious treatments depending on the subject's level of fVEGF.

### CROSS-REFERENCE TO RELATED APPLICATIONS

This application is a U.S. National Stage under 35 U.S.C. § 371 of International Application No. PCT/US2024/022928, filed Apr. 4, 2024, which claims benefit under 35 U.S.C. § 119 (e) of U.S. Provisional Application No. 63/459,399 filed Apr. 14, 2023 and U.S. Provisional Application No. 63/613,857 filed Dec. 22, 2023, the contents of each of which are incorporated herein by reference in their entireties.

### GOVERNMENT SUPPORT

This invention was made with government support under Grant Number HL119145, awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created on Jul. 9, 2024, is named “701039-000115WOPT_SL.xml” and is 16,384 bytes in size.

### TECHNICAL FIELD

The technology described herein relates to the treatment of inflammation.

### BACKGROUND

Neutrophils and other inflammatory cells play a primary role in disease progression by responding to direct and indirect insults to tissue injury and by promoting inflammation through secretion of inflammatory cytokines and proteases. While inflammation can be part of the healing process, it is dysregulated in many conditions and results in slowed healing and/or gives rise to or is a symptom of a number of inflammatory conditions and diseases. Effective ways to detect and treat inflammation are needed.

### SUMMARY

Vascular Endothelial Growth Factor (VEGF) is a ubiquitous signaling molecule that plays a key role in maintaining and promoting cell and tissue health. The inventors have discovered that when VEGF is cleaved to provide fragmented VEGF (fVEGF), the fVEGF is an extremely powerful attractant of neutrophils, which can contribute to inflammation. Further, the inventors have demonstrated that PR1P, a 12-amino acid peptide that binds to and upregulates VEGF, stabilizes VEGF from degradation and thereby limits the production of VEGF degradation products (fragmented VEGF (fVEGF)). Accordingly, provided herein are diagnostic and therapeutic treatments relating to the treatment of inflammation.

In one aspect of any of the embodiments, described herein is a peptide comprising the sequence of SEQ ID NO: 1 (DRVQRQTTTVVA). In some embodiments of any of the aspects, the peptide consists of the sequence of SEQ ID NO: 1.

In one aspect of any of the embodiments, described herein is a pharmaceutical composition comprising a peptide described herein and a pharmaceutically acceptable excipient.

In one aspect of any of the embodiments, described herein is a method of treating inflammation in a subject in need thereof, the method comprising administering an anti-inflammatory or anti-infective treatment to a subject with an increased level of fragmented VEGF (fVEGF). In some embodiments of any of the aspects, the subject is a subject determined to have an increased level of fragmented VEGF (fVEGF). In some embodiments of any of the aspects, the method further comprises a first step of detecting or measuring the level of fragmented VEGF (fVEGF) in the subject or a sample obtained from the subject. In some embodiments of any of the aspects, the anti-inflammatory reduces the rate of formation of fVEGF. In some embodiments of any of the aspects, the anti-inflammatory is a peptide or pharmaceutical composition described herein.

In one aspect of any of the embodiments, described herein is a method of treating inflammation or infection in a subject in need thereof, the method comprising administering a peptide or pharmaceutical composition described herein to the subject.

In some embodiments of any of the aspects, the fVEGF is VEGF₁₆₅. In some embodiments of any of the aspects, the level of fVEGF is the level of fVEGF in the blood of the subject, or a blood sample of the subject. In some embodiments of any of the aspects, the level of fVEGF is the level of fVEGF in the injured or infected tissue of the subject, or an injured or infected tissue sample of the subject. In some embodiments of any of the aspects, the level of fVEGF is the local level of fVEGF. In some embodiments of any of the aspects, the level of fVEGF is not the systemic level of fVEGF. In some embodiments of any of the aspects, the level of fVEGF is the level of fVEGF at a site of injury and/or infection. In some embodiments of any of the aspects, the level of fVEGF is the level of fVEGF in the blood, serum, plasma, eye humor, saliva, urine, bronchoalveolar lavage fluid, cerebrospinal fluid, or exhaled breath condensate of the subject; or a blood, serum, plasma, eye humor, saliva, urine, bronchoalveolar lavage fluid, cerebrospinal fluid, or exhaled breath condensate sample of the subject. In some embodiments of any of the aspects, the fVEGF comprises, consists of, or consists essentially of:

(SEQ ID NO: 2)
ARQENPCGPCSERRKHLVQDPQTCKCSCKNTDSRCKARQLELNERTCR
CDKPRR.

### BRIEF DESCRIPTION OF THE DRAWINGS

FIGS. 1A-1B-IL-8 and fVEGF increase human neutrophil migration in vitro. FIG. 1A) Bar graph showing the quantification of neutrophils (#neutrophils/region of interest (ROI)) during Transwell Plate Migration assay in which neutrophil migration (2h) was in response to indicated stimulants. fVEGF was commercially synthesized based on a published amino acid sequence (1):

ARQENPCGPCSERRKHLVQDPQTCKCSCKNTDSRCKARQLELNERTCRCDKPRR (SEQ ID NO: 2), derived from VEGF165 attracts human neutrophils similarly to IL-8, which is known as one of the most potent chemoattractants to neutrophils. VEGF+elastase and VEGF+elastaste+PR1P were products of 30 min incubation of VEGF and elastase in the presence or absence of PR1P, respectively. n=6 repeated 3 times. FIG. 1B Data are mean±/−SEM. * p<0.005.

FIGS. 2A-2F: PR1P inhalation reduces neutrophil migration into lungs and lung inflammation following bleomycin, LPS, and HCL injury in mice. FIG. 2A) Bar graph showing percentage of neutrophils present (% of total cells in region of interest (ROI)) in BALF following IT administration of acid (HCl) and daily inhalation of PR1P or scrambled peptide (SP). n=8 repeated 3 times. * p<0.023. FIG. 2B) Representative DIFF-QUIK stained cells from murine BALF described in FIG. 2A showing reduced neutrophils (black arrows) following PR1P treatment (25%) compared to SP (9%). FIG. 2C) Bar graph showing percentage of neutrophils present (% of total cells in ROI) in BALF following IT administration of bleomycin and daily inhalation of PR1P or scrambled peptide (SP). n=7 repeated 3 times, *p<0.02. FIG. 2D) Representative FACS analysis of cells from whole lung digests from mice treated with PR1P or SP after LPS injury showing reduced presence 24 h after injury of neutrophils following PR1P inhalation. FIG. 2E) Bar graph showing quantification of neutrophils (% of total cells) in whole lung digests from FACS experiments described in FIG. 2D. n=8, repeated 3 times. * p<0.02. FIG. 2F) Bar graph showing quantification (Optical Density (OD) at 405 nm) of indicated cytokines or myeloperoxidase (MPO) activity in BALF following bleomycin injury in experiments described in FIG. 2A. All data are mean±SEM. N=5. * p<0.004, ** p<0.05.

FIG. 3. PR1P attenuates neutrophil migration in vitro by preventing VEGF degradation. Bar graph showing the quantification of neutrophils (#Neutrophils/Region of Interest (ROI)) during Transwell Plate Migration assay in which neutrophil migration (2h) was in response to indicated stimulants. ‘fVEGF’ was commercially synthesized based on a published amino acid sequence (20). ‘VEGF+Elastase’ and ‘VEGF+Elastase+PR1P’ were products of 30 min incubation of VEGF and elastase in presence or absence of PR1P, respectively. (n=6 repeated 3 times). Data are mean+/− SEM. * p<0.004.

FIGS. 4A-4F. PR1P inhalation reduces neutrophil migration into lungs and lung inflammation following bleomycin, LPS and HCL injury in mice. (FIG. 4A) Bar graph showing percentage of neutrophils present (% of total cells in region of interest (ROI)) in BALF following IT administration of acid (HCl) and daily inhalation of PR1P or scrambled peptide (SP). n=8 repeated 3 times. * p<0.023 (FIG. 4B) Representative Diff-Quik stained cells from murine BALF described in (A) showing reduced neutrophils (black arrows) following PR1P treatment (25%) compared to SP (9%). (FIG. 4C) Bar graph showing percentage of neutrophils present (% of total cells in ROI) in BALF following IT administration of Bleomycin and daily inhalation of PR1P or scrambled peptide (SP). n=7 repeated 3 times *p<0.02. (FIG. 4D) Representative FACS analysis of cells from whole lung digests from mice treated with PR1P or SP after LPS injury showing reduced presence 24 h after injury of neutrophils following PR1P inhalation. (FIG. 4E) Bar graph showing quantification of neutrophils (% of total cells) in whole lung cell digests from FACS experiments described in (FIG. 4D) n=8, repeated 3 times *p<0.02. (FIG. 4F) Bar graph showing quantification (Optical Density (OD) at 405 nm) of indicated cytokines or myeloperoxidase (MPO) activity in BALF following bleomycin injury in experiments described in (FIG. 4A) All data are mean±SEM. n=5. * p<0.004, ** p<0.05.

FIGS. 5A-5D. PR1P improves disease outcome parameters in TNBS-induced colitis in rats. (FIG. 5A) Line graph showing average daily weights of rats treated with daily low-dose PR1P (0.5 mg/kg IP, TNBS+PR1P LO) or high-dose PR1P (5 mg/kg IP, TNBS+PR1P HI) or prednisolone (10 mg/kg po, TNBS+Prednisolone) following induction of colitis from administration of intra-rectal TNBS. Sham animals received intra-rectal 50% ethanol (vehicle) to induce colitis and no treatments. TNBS group (+TNBS) received daily treatment with vehicles IP or po. * p<0.001 vs +TNBS group. (FIGS. 5B-5D) Bar graphs showing average colon weight, colon length, length/weight ratio and colon wall thickness (FIG. 5B), *p<0.03, ** p<0.004), ulcer length and colon wall thickness (FIG. 5C, *p<0.005) and average scores for adhesions, strictures, ulcer, wall thickness and colonic score using scoring system described in methods (FIG. 5D, *p<0.05, ** p<0.009) from indicated treatment groups at the end of experiments described in (FIG. 5A). Data in (FIGS. 5A-5D) are mean±SEM. n=8 animals per group. In FIGS. 5B-5D, the first series is-TNBS, the second series is +TNBS, the third series is +TNBS/PR1P LO, the fourth series is +TNBS/PR1P HI, and the firth series is +TNBS/Prednisolone.

FIGS. 6A-6C: PR1P mediates inflammation in TNBS-induced colitis in rats. (FIGS. 6A-6C) Bar graphs showing quantification of IL-1β (FIG. 6A, *p<0.03, ** p<0.003), IL-6 (FIG. 6B, *p<0.0001) and TNF-α (FIG. 6C, *p<0.04) in plasma on day 7 in rats treated with daily low-dose PR1P (0.5 mg/kg IP, PR1P-LO) or high-dose PR1P (5 mg/kg IP, PR1P-HI) or prednisolone (10 mg/kg) following induction of colitis from administration of intra-rectal TNBS. Control rats were treated with vehicle only. Data are mean±SEM. n=8 animals per group.

FIGS. 7A-7E: PR1P reduces disease and inflammation on histopathology in TNBS-induced colitis in rats. (FIG. 7A) Bar graphs showing Average Scores (Score) of indicated lesions from histology sections of the colon of rats 7 days after intra-rectal administration of TNBS (or carrier) and daily treatment of PR1P (0.5 or 5 mg/kg IP) or Prednisolone (10 mg/kg po) as indicated (See Methods), Data are mean±SEM. * p<0.04, ** p<0.005. (n=8). In FIG. 7A the first series is-TNBS, the second series is +TNBS, the third series is +TNBS/PR1P LO, the fourth series is +TNBS/PR1P HI, and the firth series is +TNBS/Prednisolone. (FIGS. 7B-7E) Representative images of H&E stained colon sections (100×) obtained from rats on day 7 following experiments described in (FIG. 7A). (FIG. 7B) Representative image from Naïve animal. No histologic lesions observed in captured region. The mucosal glands (M) are within normal limits, as are the submucosa (SM), tunica muscularis externa (TM), and the serosa(S). (FIG. 7C) Representative image from animal with TNBS-induced colitis treated with vehicle (TNBS+Vehicle). No normal mucosa remains in captured region; all mucosa exhibits coagulative necrosis (NM: necrotic mucosa), with complete loss of the surface epithelium, consistent with erosion. A thick band of inflammatory cells (*) are visible at the mucosal-submucosal (SM) border. The submucosa is edema-expanded and contains abundant hemorrhage and erythrophagocytic macrophages (black arrowheads). Inflammatory cells (*) also extend transmurally through the tunica musuclaris (TM) and serosa(S) into the serosal granulation tissue visible at the bottom of the image. (FIG. 7D) Representative image from animal with TNBS-induced colitis treated with high dose PR1P (5 mg/kg, TNBS+PR1P HI). The right side of the image exhibits inflammatory cell infiltration and gland loss (**) with surface epithelial loss (erosion; black arrows). The adjacent mucosa (M) is hyperplastic, with elongated colonic glands observed. The submucosa (SM) is expanded by edema and inflammatory cell infiltration (*). Very mild extension of inflammation (*) is seen through the tunica muscularis (TM) and into the serosa(S). (FIG. 7E) Representative image from animal with TNBS-induced colitis treated with Prednisolone (TNBS+Prednisolone). Inflammatory cell infiltration with gland loss (**) and erosion (black arrows) are mild; the adjacent mucosa (M) exhibits reactive hyperplasia. There is expansion of the submucosa (SM) by edema (clear/pale area) and inflammatory cell infiltration (*). Inflammatory cells are generally limited to the mucosal and submucosa with minimal involvement of the tunica muscularis externa (TM) and serosa(S).

### DETAILED DESCRIPTION

As described herein, the inventors have discovered that fVEGF is a surprisingly strong neutrophil attractant, and it is contemplated herein that fVEGF could be a key early driver of inflammatory processes. Accordingly, described herein are diagnostic and therapeutic methods that relate to targeting patients with high fVEGF levels for anti-inflammatory treatment. Additionally, the inventors demonstrated that PR1P is an effective inhibitor of fVEGF generation and therefore described herein are therapeutic methods relating to the administration of PR1P.

In one aspect of any of the embodiments, described herein is a peptide comprising the sequence of SEQ ID NO: 1 (DRVQRQTTTVVA). As used herein, “PR1P” or “Prominin-1 Peptide” refers to the peptide having the sequence of SEQ ID NO: 1.

In some of the embodiments of any of the aspects, described herein is a peptide comprising at least one PR1P domain. In some of the embodiments of any of the aspects, described herein is a peptide comprising at least one PR1P domain, each PR1P domain comprising the sequence of SEQ ID NO: 1. In some of the embodiments of any of the aspects, described herein is a peptide consisting essentially of at least one PR1P domain, each PR1P domain comprising the sequence of SEQ ID NO: 1. In some of the embodiments of any of the aspects, described herein is a peptide comprising at least one PR1P domain, each PR1P consisting of the sequence of SEQ ID NO: 1.

In some of the embodiments of any of the aspects, described herein is a peptide comprising a PR1P domain, the PR1P domain comprising the sequence of SEQ ID NO: 1. In some of the embodiments of any of the aspects, described herein is a peptide comprising a PR1P domain, the PR1P domain consisting essentially of the sequence of SEQ ID NO: 1. In some of the embodiments of any of the aspects, described herein is a peptide comprising a PR1P domain, the PR1P domain consisting of the sequence of SEQ ID NO: 1.

In some of the embodiments of any of the aspects, described herein is a peptide consisting of a PR1P domain, the PR1P domain comprising the sequence of SEQ ID NO: 1. In some of the embodiments of any of the aspects, described herein is a peptide consisting of a PR1P domain, the PR1P domain consisting essentially of the sequence of SEQ ID NO: 1. In some of the embodiments of any of the aspects, described herein is a peptide consisting of a PR1P domain, the PR1P domain consisting of the sequence of SEQ ID NO: 1.

In some embodiments of any of the aspects, the at least one PR1P domain comprises, consists of, or consists essentially of a sequence with at least 80%, at least 85%, at least 90%, at least 95%, at least 98%, at least 99%, or greater sequence identity to SEQ ID NO: 1. In some embodiments of any of the aspects, the at least one PR1P domain comprises, consists of, or consists essentially of a sequence with at least 80% sequence identity to SEQ ID NO: 1. In some embodiments of any of the aspects, the at least one PR1P domain comprises, consists of, or consists essentially of a sequence with at least 95% sequence identity to SEQ ID NO: 1.

In some embodiments of any of the aspects, the at least one PR1P domain comprises, consists of, or consists essentially of a sequence with at least 80%, at least 85%, at least 90%, at least 95%, at least 98%, at least 99%, or greater sequence identity to SEQ ID NO: 1, wherein the residue corresponding to the first residue of SEQ ID NO: 1 is D and the residue corresponding to the fifth residue of SEQ ID NO: 1 is R. In some embodiments of any of the aspects, the at least one PR1P domain comprises, consists of, or consists essentially of a sequence with at least 80% sequence identity to SEQ ID NO: 1, wherein the residue corresponding to the first residue of SEQ ID NO: 1 is D and the residue corresponding to the fifth residue of SEQ ID NO: 1 is R. In some embodiments of any of the aspects, the at least one PR1P domain comprises, consists of, or consists essentially of a sequence with at least 95% sequence identity to SEQ ID NO: 1, wherein the residue corresponding to the first residue of SEQ ID NO: 1 is D and the residue corresponding to the fifth residue of SEQ ID NO: 1 is R.

In some embodiments of any of the aspects, the at least one PR1P domain comprises, consists of, or consists essentially of a sequence with at least 80%, at least 85%, at least 90%, at least 95%, at least 98%, at least 99%, or greater sequence identity to SEQ ID NO: 1, wherein the residue corresponding to the first residue of SEQ ID NO: 1 is D. In some embodiments of any of the aspects, the at least one PR1P domain comprises, consists of, or consists essentially of a sequence with at least 80% sequence identity to SEQ ID NO: 1, wherein the residue corresponding to the first residue of SEQ ID NO: 1 is D. In some embodiments of any of the aspects, the at least one PR1P domain comprises, consists of, or consists essentially of a sequence with at least 95% sequence identity to SEQ ID NO: 1, wherein the residue corresponding to the first residue of SEQ ID NO: 1 is D.

In some embodiments of any of the aspects, the at least one PR1P domain comprises, consists of, or consists essentially of a sequence with at least 80%, at least 85%, at least 90%, at least 95%, at least 98%, at least 99%, or greater sequence identity to SEQ ID NO: 1, wherein the residue corresponding to the fifth residue of SEQ ID NO: 1 is R. In some embodiments of any of the aspects, the at least one PR1P domain comprises, consists of, or consists essentially of a sequence with at least 80% sequence identity to SEQ ID NO: 1, wherein the residue corresponding to the fifth residue of SEQ ID NO: 1 is R. In some embodiments of any of the aspects, the at least one PR1P domain comprises, consists of, or consists essentially of a sequence with at least 95% sequence identity to SEQ ID NO: 1, wherein the residue corresponding to the fifth residue of SEQ ID NO: 1 is R.

In some embodiments of any of the aspects, the at least one PR1P domain comprises, consists of, or consists essentially of a sequence with at least 80%, at least 85%, at least 90%, at least 95%, at least 98%, at least 99%, or greater sequence identity to SEQ ID NO: 1. In some embodiments of any of the aspects, the at least one PR1P domain comprises, consists of, or consists essentially of a sequence with at least 80% sequence identity to SEQ ID NO: 1. In some embodiments of any of the aspects, the at least one PR1P domain comprises, consists of, or consists essentially of a sequence with at least 95% sequence identity to SEQ ID NO: 1.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

In some embodiments of any of the aspects, a peptide described herein does not comprise the sequence of any of SEQ ID NOs: 3-11. In some embodiments of any of the aspects, a peptide described herein does not comprise the sequence of SEQ ID NO: 3. In some embodiments of any of the aspects, a peptide described herein does not comprise the sequence of SEQ ID NO: 4. In some embodiments of any of the aspects, a peptide described herein does not comprise the sequence of SEQ ID NO: 5. In some embodiments of any of the aspects, a peptide described herein does not comprise the sequence of SEQ ID NO: 6. In some embodiments of any of the aspects, a peptide described herein does not comprise the sequence of SEQ ID NO: 7. In some embodiments of any of the aspects, a peptide described herein does not comprise the sequence of SEQ ID NO: 8. In some embodiments of any of the aspects, a peptide described herein does not comprise the sequence of SEQ ID NO: 10. In some embodiments of any of the aspects, a peptide described herein does not comprise the sequence of SEQ ID NO: 11.

SEQ ID NO: 3
malvlgslll lglcgnsfsg gqpsstdapk awnyelpatn yetqdshkag
pigilfelvh
iflyvvqprd fpedtlrkfl qkayeskidy dkivyyeagi ilccvlgllf
iilmplvgyf
fcmcrconkc ggemhqrqke ngpflrkcfa isllviciii sigifygfva
nhqvrtrikr
srkladsnfk dlrtllnetp eqikyilaqy nttkdkaftd lnsinsvlgg
gildrlrpni
ipvldeiksm ataiketkea lenmnstlks lhqqstqlss sltsvktslr
sslndplclv
hpssetcnsi rlslsqlnsn pelrqlppvd aeldnvnnvl rtdldglvqq
gyqslndip
SEQ ID NO: 4
gikrvlnsi gsdidnvtqr lpiqdilsaf svyvnntesy ihrnlptlee
ydsywwlggl vicslltliv ifyylgllcg vcgydrhatp ttrgcvsntg
gvflmvgvgl
sflfcwilmi ivvltfvfga nveklicepy tskelfrvld tpyllnedwe
yylsgklfnk
skmkltfeqv ysdckknrgt ygtlhlqnsf nisehlnine htgsissele
slkvnlnifl
lgaagrknlq dfaacgidrm nydsylaqtg kspagvnlls faydleakan
slppgnlrns
lkrdaqtikt ihqqrvlpie qslstlyqsv kilqrtgngl lervtrilas
ldfaqnfitn
ntssviieet kkygrtiigy fehylqwief sisekvasck pvataldtav
dvflcsyiid
plnlfwfgig katvillpal ifavklakyy rrmdsedvyd dvetipmknm
engnngyhkd
hvygihnpvm tspsqh
SEQ ID NO: 5
malvlgslll lglcgnsfsg gqpsstdapk awnyelpatn yetqdshkag
pigilfelvh
iflyvvqprd fpedtlrkfl qkayeskidy dkpetvilgl kivyyeagii
lccvlgllfi
ilmplvgyff cmcrccnkcg gemhqrqken gpflrkcfai sllviciiis
igifygfvan
hqvrtrikrs rkladsnfkd lrtllnetpe qikyilaqyn ttkdkaftdl
nsinsvlggg
ildrlrpnii pvldeiksma taiketkeal enmnstlksl hqqstqlsss
ltsvktslrs
slndplclvh pssetcnsir lslsqlnsnp elrqlppvda eldnvnnvlr
tdldglvqqg
yqslndip
SEQ ID NO: 6
gikrvlnsig sdidnvtqrl piqdilsafs vyvnntesyi
hrnlptleey dsywwlgglv icslltlivi fyylgllcgv cgydrhatpt
trgcvsntgg
vflmvgvgls flfcwilmii vvltfvfgan veklicepyt skelfrvldt
pyllnedwey
ylsgklinks kmkltfeqvy sdckknrgty gtlhlqnsfn isehlnineh
tgsisseles
lkvnlnifll
gaagrknlqd faacgidrmn ydsylaqtgk spagvnllsf
aydleakans
lppgnlrnsl krdaqtikti hqqrvlpieq slstlyqsvk ilqrtgngll
ervtrilasl
dfaqnfitnn tssviieetk kygrtiigyf ehylqwiefs isekvasckp
vataldtavd
vflcsyiidp lnlfwfgigk atvillpali favklakyyr rmdsedvydd
vetipmknps
qh
SEQ ID NO: 7
gikrvlnsig sdidnvtqrl piqdilsafs vyvnntesyi
hrnlptleey dsywwlgglv icslltlivi fyylgllcgv cgydrhatpt
trgcvsntgg
vflmvgvgls flfcwilmii vvltfvfgan veklicepyt skelfrvldt
pyllnedwey
ylsgklfnks
kmkltfeqvy sdckknrgty gtlhlqnsfn isehlnineh
tgsisseles
lkvnlnifll gaagrknlqd faacgidrmn ydsylaqtgk spagvnllsf
aydleakans
lppgnlrnsl krdaqtikti hqqrvlpieq slstlyqsvk ilqrtgngll
ervtrilasl
dfaqnfitnn tssviieetk kygrtiigyf ehylqwiefs isekvasckp
vataldtavd
vflcsyiidp lnlfwfgigk atvillpali favklakyyr rmdsedvydd psqh
SEQ ID NO: 8
gikrvlnsi gsdidnvtqr lpiqdilsaf svyvnntesy ihrnlptlee
ydsywwlggl vicslltliv ifyylgllcg vcgydrhatp ttrgcvsntg
gvflmvgvgl
sflfcwilmi ivvltfvfga nveklicepy tskelfrvld tpyllnedwe
yylsgklfnk
skmkltfeqv ysdckknrgt ygtlhlqnsf nisehlnine htgsissele
slkvnlnifl
lgaagrknlq dfaacgidrm nydsylaqtg kspagvnlls faydleakan
slppgnlrns
lkrdaqtikt ihqqrvlpie qslstlyqsv kilqrtgngl lervtrilas
ldfaqnfitn
ntssvijeet kkygrtiigy fehylqwief sisekvasck pvataldtav
dvflcsyiid
plnlfwfgig katvillpal ifavklakyy rrmdsedvyd dvetipmknp
SEQ ID NO: 10
gikrvlnsi gsdidnvtqr lpiqdilsaf svyvnntesy ihrnlptlee
ydsywwlggl vicslltliv ifyylgllcg vcgydrhatp ttrgcvsntg
gvflmvgvgl
sflfcwilmi ivvltfvfga nveklicepy tskelfrvld tpyllnedwe
yylsgklfnk
skmkltfeqv ysdckknrgt ygtlhlqnsf nisehlnine htgsissele
slkvnlnifl
lgaagrknlq dfaacgidrm nydsylaqtg kspagvnlls faydleakan
slppgnlrns
lkrdaqtikt ihqqrvlpie qslstlyqsv kilqrtgngl lervtrilas
ldfaqnfitn
ntssvijeet kkygrtiigy fehylqwief sisekvasck pvataldtav
dvflcsyiid
plnlfwfgig katvillpal ifavklakyy rrmdsedvyd dpsqh
SEQ ID NO: 11
gikrvlnsig sdidnvtqrl piqdilsafs vyvnntesyi
hrnlptleey dsywwlgglv icslltlivi fyylgllcgv cgydrhatpt
trgcvsntgg
vflmvgvgls flfcwilmii vvltfvfgan veklicepyt skelfrvldt
pyllnedwey
ylsgklinks kmkltfeqvy sdckknrgty gtlhlqnsfn isehlnineh
tgsisseles
lkvnlnifll gaagrknlqd faacgidrmn ydsylaqtgk spagvnllsf
aydleakans
lppgnlrnsl krdaqtikti hqqrvlpieq slstlyqsvk ilqrtgngll
ervtrilasl
dfaqnfitnn tssviieetk kygrtiigyf ehylqwiefs isekvasckp
vataldtavd
vflcsyiidp lnlfwfgigk atvillpali favklakyyr rmdsedvydd
vetipmknme
ngnngyhkdh vygihnpvmt spsqh

In some embodiments of any of the aspects, a peptide described herein comprises one PR1P domain. In some embodiments of any of the aspects, a peptide described herein consists of one PR1P domain. In some embodiments of any of the aspects, a peptide described herein consists essentially of one PR1P domain.

A peptide described herein can comprise multiple PR1P domains, e.g., 2, 3, 4, 5, or more PR1P domains. In some embodiments of any of the aspects, a peptide described herein comprises two or more PR1P domains. In some embodiments of any of the aspects, a peptide described herein consists of two or more PR1P domains. In some embodiments of any of the aspects, a peptide described herein consists essentially of two or more PR1P domains.

In some embodiments of any of the aspects, a peptide described herein comprises two PR1P domains. In some embodiments of any of the aspects, a peptide described herein consists of two PR1P domains. In some embodiments of any of the aspects, a peptide described herein consists essentially of two PR1P domains. In some embodiments of any of the aspects, a peptide described herein comprises three PR1P domains. In some embodiments of any of the aspects, a peptide described herein consists of three PR1P domains. In some embodiments of any of the aspects, a peptide described herein consists essentially of three PR1P domains. In some embodiments of any of the aspects, a peptide described herein comprises four PR1P domains. In some embodiments of any of the aspects, a peptide described herein consists of four PR1P domains. In some embodiments of any of the aspects, a peptide described herein consists essentially of four PR1P domains.

In some embodiments of any of the aspects, the peptide consists of the at least one PR1P domain. In some embodiments of any of the aspects, the peptide consists essentially of the at least one PR1P domain.

In some embodiments of any of the aspects, a peptide described herein is an engineered peptide. In some embodiments of any of the aspects, a peptide described herein comprises a non-naturally occurring sequence. In some embodiments of any of the aspects, a peptide described does not comprise a wild-type sequence, e.g., human sequence or a homolog thereof.

The peptides described herein can comprise modifications, e.g., chemical moieties added or substituted to the polypeptide structure as defined solely by the sequence. Exemplary, but non-limiting modifications include acetylation, methyl esterification, amidation, carboxylation, N-ethylamidation, or addition of pyroglutamate. In some embodiments of any of the aspects, the peptide described herein further comprises a N-terminus modification. In some embodiments of any of the aspects, the peptide described herein further comprises at least one N-terminus modification. In some embodiments of any of the aspects, the peptide described herein further comprises a C-terminus modification. In some embodiments of any of the aspects, the peptide described herein further comprises at least one C-terminus modification.

In some embodiments, the technology described herein relates to a pharmaceutical composition comprising a peptide as described herein, and optionally a pharmaceutically acceptable carrier. In some embodiments, the active ingredients of the pharmaceutical composition comprise a peptide as described herein. In some embodiments, the active ingredients of the pharmaceutical composition consist essentially of a peptide as described herein. In some embodiments, the active ingredients of the pharmaceutical composition consists of a peptide as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as peptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g., a peptide as described herein.

In some embodiments, the pharmaceutical composition comprising a peptide as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a peptide as disclosed within are well known to those skilled in the art. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a peptide as disclosed herein can also be incorporated into the parenteral dosage forms of the disclosure, including conventional and controlled-release parenteral dosage forms.

Pharmaceutical compositions comprising a peptide as described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the peptide as described herein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

As described herein, the instant peptides inhibit VEGF fragmentation and neutrophil migration, e.g., inhibits inflammation or promotes resolution of inflammation. The peptides are therefore contemplated for use in treating inflammation.

In one aspect of any of the embodiments, described herein is a method of treating inflammation or infection in a subject in need thereof, the method comprising administering the peptide or pharmaceutical composition described herein to the subject.

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death. Inflammation can include both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

An inflammatory condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments, the inflammatory condition is associated with wound healing. In some embodiments, the inflammation to be treated according to the methods described herein can be skin inflammation; inflammation caused by substance abuse or drug addiction;

inflammation associated with infection; inflammation of the cornea; inflammation of the retina; inflammation of the spinal cord; inflammation associated with organ regeneration; and pulmonary inflammation.

In some embodiments, an inflammatory condition can be an autoimmune disease. Non-limiting examples of autoimmune diseases can include: Type 1 diabetes; systemic lupus erythematosus; rheumatoid arthritis; psoriasis; inflammatory bowel disease; Crohn's disease; and autoimmune thyroiditis.

In some embodiments, a subject in need of treatment for inflammation can be a subject having, or diagnosed as having temporomandibular joint disorders; COPD; smoke-induced lung injury; renal dialysis associated disorders; spinal cord injury; graft vs. host disease; bone marrow transplant or complications thereof; infection; trauma; pain; incisions; surgical incisions; a chronic pain disorder; a chronic bone disorder; mastitis; and joint disease. In some embodiments, trauma can include battle-related injuries or tissue damage occurring during a surgery. Smoke-induced lung injury can result from exposure to tobacco smoke, environmental pollutants (e.g. smog or forest fires), or industrial exposure. By way of non-limiting example, inflammatory conditions can be inflammatory conditions of the skin, such as Sweet's syndrome, pyoderma gangrenosum, subcorneal pustular dermatosis, erythema elevatum diutinum, Behcet's disease or acute generalized exanthematous pustulosis, a bullous disorder, psoriasis, a condition resulting in pustular lesions, acne, acne vulgaris, dermatitis (e.g. contact dermatitis, atopic dermatitis, seborrheic dermatitis, eczematous dermatitides, eczema craquelee, photoallergic dermatitis, phototoxicdermatitis, phytophotodermatitis, radiation dermatitis, stasis dermatitis or allergic contact dermatitis), eczema, ulcers and erosions resulting from trauma, burns, ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin, cutaneous atrophy resulting from the topical use of corticosteroids, and inflammation of mucous membranes (e.g., cheilitis, chapped lips, nasal irritation, mucositis and vulvovaginitis).

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions. By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex. By way of non-limiting example, inflammatory conditions can be inflammatory conditions of the cardiovascular system, such as coronary infarct damage, peripheral vascular disease, myocarditis, vasculitis, revascularization of stenosis, artherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia. By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection. By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute hemorrhagic leucoencephalitis (Hurst's disease); human T-lymphotropic virus type-1 associated myelopathy/tropical spactic paraparesis; Devic's disease; human immunodeficiency virus encephalopathy; human immunodeficiency virus vacuolar myelopathy; peripheral neuropathies; Guillain-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS). Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopeniaurpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediaterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organising pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines. In some embodiments, an inflammatory condition is associated with an infection, e.g., viral, bacterial, fungal, parasite or prion infections. In some embodiments, an inflammatory condition is associated with an allergic response. In some embodiments, an inflammatory condition is associated with a pollutant (e.g., asbestosis, silicosis, or berylliosis).

In some embodiments, the inflammatory condition can be a local condition, e.g., a rash or allergic reaction. In some embodiments, the inflammation is associated with a wound.

In some embodiments, the technology described herein relates to methods of promoting wound healing. As used herein, “wound” refers broadly to injuries to an organ or tissue of an organism that typically involves division of tissue or rupture of a membrane (e.g., skin), due to external violence, a mechanical agency, or infectious disease. A wound can be an epithelial, endothelial, connective tissue, ocular, or any other kind of wound in which the strength and/or integrity of a tissue has been reduced, e.g. trauma has caused damage to the tissue. The term “wound” encompasses injuries including, but not limited to, lacerations, abrasions, avulsions, cuts, burns, velocity wounds (e.g., gunshot wounds), penetration wounds, puncture wounds, contusions, diabetic wounds, hematomas, tearing wounds, and/or crushing injuries. In one aspect, the term “wound” refers to an injury to the skin and subcutaneous tissue initiated in any one of a variety of ways (e.g., pressure sores from extended bed rest, wounds induced by trauma, cuts, ulcers, burns and the like) and with varying characteristics. As used herein, the term “wound healing” refers to a process by which the body of a wounded organism initiates repair of a tissue at the wound site (e.g., skin). The wounds healing process requires, in part, angiogenesis and revascularization of the wounded tissue. Wound healing can be measured by assessing such parameters as contraction, area of the wound, percent closure, percent closure rate, and/or infiltration of blood vessels as known to those of skill in the art. In some embodiments, the particles and compositions described herein can be applied topically to promote wound healing.

The compositions and methods described herein can be administered to a subject having or diagnosed as having one of the conditions described herein, e.g., inflammation In some embodiments, the methods described herein comprise administering an effective amount of compositions described herein, to a subject in order to alleviate a symptom of a condition described herein. In some embodiments of any of the aspects, a therapeutically effective dose of the composition is administered. As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to parenteral, intravenous, intramuscular, subcutaneous, transdermal, airway (aerosol), pulmonary, cutaneous, injection, or intratumoral administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g. for neutrophil migration, fVEGF levels, inflammatory markers, among others. The dosage can be determined by a physician and adjusted, as necessary, to suit observed effects of the treatment.

In some embodiments of any of the aspects, a treatment described herein is administered as a monotherapy, e.g., another treatment for the condition is not administered to the subject.

By way of non-limiting example, if a subject is to be treated for inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs-such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g., endorphins, enkephalins and dynorphin), steroids, analgesics, barbiturates, oxycodone, morphine, lidocaine and the like.

In certain embodiments, an effective dose of a treatment as described herein can be administered to a patient once. In certain embodiments, an effective dose of a treatment described herein can be administered to a patient repeatedly. In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition or treatment as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the composition. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition or treatment as described herein can be administered over a period of time, such as over a 5 minute, 10 minute, 15 minute, 20 minute, or 25 minute period.

The dosage ranges for the administration of the composition and treatments described herein, according to the methods described herein depend upon, for example, the potency of the active ingredients, and the extent to which symptoms, markers, or indicators of a condition described herein are desired to be reduced, for example the percentage reduction desired for neutrophil levels or migration or the extent to which, for example, wound healing are desired to be induced. The dosage should not be so large as to cause adverse side effects, such as immunosuppression. Generally, the dosage will vary with the age, condition, and sex of the patient and can be determined by one of skill in the art. The dosage can also be adjusted by the individual physician in the event of any complication.

As described herein, a subject treated in accordance with the methods described herein can be a subject with an increased level of fragmented VEGF (fVEGF). Neutrophil derived proteases such as elastase and plasmin cleave VEGF (20-22) into a smaller VEGF isomer (fragmented VEGF (fVEGF)) with altered VEGF signaling. That is, fVEGF is a by-product of VEGF degradation. In some embodiments, fVEGF comprises, consists of, or consists essentially of:

ARQENPCGPCSERRKHLVQDPQTCKCSCKNTDSRCKARQLELNERTCRCDKPRR (SEQ ID NO: 2). In some embodiments, fVEGF comprises SEQ ID NO: 2. In some embodiments, fVEGF consists of SEQ ID NO: 2. In some embodiments, the fVEGF is VEGF₁₆₅.

In some embodiments, the level of fVEGF is the level of fVEGF in the blood of the subject, or a blood sample of the subject. In some embodiments, the level of fVEGF is the level of fVEGF in the injured or infected tissue of the subject, or an injured or infected tissue sample of the subject. In some embodiments, the level of fVEGF is the local level of fVEGF. In some embodiments, the level of fVEGF is not the systemic level of fVEGF. In some embodiments, the level of fVEGF is the level of fVEGF at a site of injury and/or infection.

In some embodiments, the level of fVEGF is the level of fVEGF in the blood, serum, plasma, eye humor, saliva, urine, bronchoalveolar lavage fluid, cerebrospinal fluid, or exhaled breath condensate of the subject; or a blood, serum, plasma, eye humor, saliva, urine, bronchoalveolar lavage fluid, cerebrospinal fluid, or exhaled breath condensate sample of the subject.

Accordingly, in one aspect of any of the embodiments, described herein is a method of treating inflammation in a subject in need thereof, the method comprising administering an anti-inflammatory or anti-infective treatment to a subject determined to have a level of fVEGF that is increased relative to a reference. In one aspect of any of the embodiments, described herein is a method of determining a patient is in need of treatment for inflammation, the method comprising determining the level of fVEGF in a sample obtained from the subject and determining that the patient is in need of treatment for inflammation if the level of fVEGF is increased relative to a reference.

In one aspect of any of the embodiments, described herein is a method of treating inflammation in a subject in need thereof, the method comprising: a) determining the level of fVEGF in a sample obtained from a subject; and b) administering an anti-inflammatory or anti-infective treatment to the subject if the level of fVEGF is increased relative to a reference. In some embodiments of any of the aspects, the method comprises administering an anti-inflammatory or anti-infective treatment to a subject previously determined to have a level of fVEGF that is increased relative to a reference. In some embodiments of any of the aspects, described herein is a method of treating inflammation in a subject in need thereof, the method comprising: a) first determining the level of fVEGF in a sample obtained from a subject; and b) then administering an anti-inflammatory or anti-infective treatment to the subject if the level of fVEGF is increased relative to a reference.

In some embodiments of any of the aspects, the step of determining if the subject has an increased level of fVEGF can comprise i) obtaining or having obtained a sample from the subject and ii) performing or having performed an assay on the sample obtained from the subject to determine/measure the level of fVEGF in the subject. In some embodiments of any of the aspects, the step of determining if the subject has an increased level of fVEGF can comprise performing or having performed an assay on a sample obtained from the subject to determine/measure the level of fVEGF in the subject. In some embodiments of any of the aspects, the step of determining if the subject has an increased level of fVEGF can comprise ordering or requesting an assay on a sample obtained from the subject to determine/measure the level of fVEGF in the subject. In some embodiments of any of the aspects, the step of determining if the subject has an increased level of fVEGF can comprise receiving the results of an assay on a sample obtained from the subject to determine/measure the level of fVEGF in the subject. In some embodiments of any of the aspects, the step of determining if the subject has an increased level of fVEGF can comprise receiving a report, results, or other means of identifying the subject as a subject with an increased level of fVEGF. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

In some embodiments of any of the aspects, measurement of the level of a target and/or detection of the level or presence of a target, e.g., of an expression product (e.g., fVEGF polypeptide) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. a fVEGF polypeptide can comprise contacting a sample obtained from a subject with a reagent (e.g., a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a polypeptide can be detected in a subject by introducing into a subject a labeled anti-peptide antibody and other types of detection agent. For example, the antibody can be labeled with a detectable marker whose presence and location in the subject is detected by standard imaging techniques. Polypeptide levels can be detected by, e.g., immunohistochemistry (“IHC”), immunocytochemistry (“ICC”) Western blot analysis, two-dimensional gel electrophoresis systems, radioimmunoassay (RIA), ELISA (enzyme linked immunosorbent assay), “sandwich” immunoassays, immunoprecipitation assays, immunodiffusion assays, agglutination assays, e.g., latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art.

In some embodiments of any of the aspects, one or more of the reagents (e.g., an antibody reagent) described herein can comprise a detectable label and/or comprise the ability to generate a detectable signal (e.g., by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g., antibodies and nucleic acid probes) are well known in the art.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a population of subjects who do not have or are not diagnosed as having, and/or do not exhibit signs or symptoms of inflammation. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

In some embodiments of any of the aspects, the level of expression products of no more than 200 other genes is determined. In some embodiments of any of the aspects, the level of expression products of no more than 100 other genes is determined. In some embodiments of any of the aspects, the level of expression products of no more than 20 other genes is determined. In some embodiments of any of the aspects, the level of expression products of no more than 10 other genes is determined.

In some embodiments of the foregoing aspects, the expression level of a given gene can be normalized relative to the expression level of one or more reference genes or reference proteins.

In some embodiments, the reference level can be the level in a sample of similar cell type, sample type, sample processing, and/or obtained from a subject of similar age, sex and other demographic parameters as the sample/subject for which the level of fVEGF is to be determined. In some embodiments, the test sample and control reference sample are of the same type, that is, obtained from the same biological source, and comprising the same composition, e.g., the same number and type of cells.

The term “sample” or “test sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

The test sample can be obtained by removing a sample from a subject, but can also be accomplished by using a previously isolated sample (e.g., isolated at a prior timepoint and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having) inflammation or a subject at risk of or at increased risk of developing inflammation as described elsewhere herein.

Anti-inflammatory agents are known in the art. Exemplary anti-inflammatoires include, but are not limited to, non-steroidal anti-inflammatory drugs (NSAIDs-such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; opiates (e.g. endorphins, enkephalins, and dynorphin), steroids, analgesics, barbiturates, oxycodone, morphine, lidocaine, and the like. In some embodiments, the anti-inflammatory agent can be a steroid (e.g., a corticosteroid or glucocorticoid); a calcineurin inhibitor (e.g. cyclosporine, tacrolimus, pimecrolimus, or FK506); an mTOR inhibitor (e.g., everolimus, temsirolimus, rapamycin, deforolimus, TOP216, OSI-027, TAFA93, nab-rapamycin, tacrolimus, biolimus, CI-779, ABT-578, AP-23675, BEZ-235, QLT-0447, ABI-009, BC-210, salirasib, AP-23841, AP-23573, KU-0059475, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin, 40-O-(2-hydroxyethyl)-rapamycin, 32-deoxorapamycin; 16-pent-2-ynyloxy-32 (S)-dihydrorapamycin; socalledrapalogs; AP23464; PI-103, PP242, PP30, Torin1; and derivatives or pharmaceutically acceptable salts thereof as well as and compounds described in, e.g. U.S. Patent Publications 2011/0178070; 2011/0021515; 2007/0112005; 2011/0054013; International Patent Publications WO98/02441; WO01/14387; WO99/15530; WO07/135411; WO03/64383; WO96/41807; WO95/16691; WO94/09010; European Patent No. EP1880723; and U.S. Pat. Nos. 8,163,775; 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; which are incorporated by reference herein in their entireties.); rapamycin (sirolimus) or an analogue thereof (e.g. everolimus, temsirolimus, ridaforolimus, deforolimus); or an anti-prolferative agent (e.g. mycophenoloate moefitil, azathioprine). In some embodiments, the mTOR inhibitor can be rapamycin or an analogue thereof, e.g. everolimus, temsirolimus, ridaforolimus, or deforolimus. Anti-proliferative agents can include, by way of non-limiting example, alkylating agents (e.g. cyclophosphamide, platinum compounds, and nitrosoureass), antimetabolites (e.g. methotrexate, azathioprine, mercaptopurine, fluorouracil, etc), and cytotoxic antibiotics (e.g., dactinomycin, anthracyclines, mitomycin C, bleomycin, and mithramycin).

Anti-infectives are known in the art and include, by way of non-limiting example antibiotics, antifungals, and antivirals.

As used herein, “antiviral” refers to any chemical or biological agent with therapeutic usefulness in the inhibition of viral transmission, activity, or replication. Categories of antivirals can include, but are not limited to entry inhibitors, uncoating inhibitors, viral synthesis inhibitors, assembly inhibitors, and release inhibitors. Exemplary, non-limiting antivirals include enfuvirtide, amantadine, rimantadine, pleconaril, acyclovir, zidovudine, lamivudine, fomivirsen, rifampicin, zanamivir, oseltamivir, peramivir, abacavir, acyclovir, adefovir, amprenavir, baloxavir marboxil, boceprevir, cobicistat, combivir, daclatasvir, doravirine, etravirine, ganciclovir, ibalizumab, letermovir, rilpivirine, simeprevir, telbivudine, and valciclovir. One of skill in the art can readily identify an antiviral agent of use e.g. see Antiviral Drugs, Wieslaw M. Kazmierski (ed.) Wiley and Sons (2011); Antiviral Drugs, John S. Driscoll. Wiley and Sons (2005); each of which is incorporated by reference herein in its entirety.

As used herein, “antibiotic” refers to any chemical or biological agent with therapeutic usefulness in the inhibition of bacterial cell growth or in killing bacteria, e.g, those that are bactericidal or bacteriostatic. Categories of antibiotics can include, but are not limited to those that target the bacterial cell wall (e.g., penicillins, cephalosporins), those that target the bacterial cell membrane (e.g., polymyxins), those that target bacterial enzymes (e.g., rifamycins, lipiarmycins, quinolones, sulfonamides), protein synthesis inhibitors (e.g., macrolides, lincosamides, and tetracyclines), aminoglycosides, cyclic lipopeptides, glycyclines, oxazolidinones, beta-lactams, and lipiarmycins. Exemplary, non-limiting antibiotics include penicillin, methicilling, nafcillin, oxacillin, cloxacillin, dicloxacillin, flucloxacillin, ampicillin, amoxicillin, pivampicillin, hetacillin, bacampicillin, metampicillin, talamipicillin, epicillin, cabenicillin, ticaricillin, temocillin, mezlocillin, piperacillin, azolocillin, clavulanic acid, sulbactam, tazobactam, cafadroxil, cephalexin, cefalotin, cefapirin, cefazolin, cefradine, cefaclor, cefonicid, cefprozil, cefuroxime, loracarbef, cefmetazole, cefotetan, cefoxitin, cefotiam, cefdinir, cefixime, cefotaxime, cefovecin, cefpodoxime, ceftibuten, ceftiofur, ceftizoxime, ceftriaxone, cefoperazone, ceftazimdime, latamoxef, cefepime, cefiderocol, cefpriome, rifampicin, rifabutin, rifapentine, rifamixin, fidaxomicin, ciproflaxicin, moxifloxacin, levofloxacin, sulfafurzole, azithromycin, clarithromycin, erythromycin, fidaxomicin, spiramycin, telihtromycin, lincomycin, clindamycin, pirlimycin, tetracycline, eravacycline, sarecycline, omadacycline, doxycycline, kanamycin, tobramycin, gentamicin, neomycin, streptomycin, vancomycin, tigecycline, linezolid, posizolid, tedizolid, radezolid, cycloserine, contezolid, and daptomycin. One of skill in the art can readily identify an antibiotic agent of use e.g. see Antibiotics in Laboratory Medicine, Victor Lorian (ed.) Wolters Kluwer; and Antibotics Manual, David Schlossberg and Rafik Samuel, John Wiley and Sons (2017); each of which is incorporated by reference herein in its entirety.

In some embodiments, the anti-inflammatory is the peptide described herein or a pharmaceutical composition comprising a peptide as described herein.

In some embodiments, the technology described herein relates to a nucleic acid encoding a peptide as described herein. In some embodiments, the nucleic acid is a cDNA.

In some embodiments, a nucleic acid encoding a peptide as described herein is comprised by a vector. In some of the aspects described herein, a nucleic acid sequence encoding a peptide, is operably linked to a vector.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as polypeptides in recoverable amounts. The precise nature of the regulatory regions needed for gene expression may vary from organism to organism, as is well known in the analogous art.

In one aspect of any of the embodiments, described herein is a cell comprising an a polypeptide as described herein, or a nucleic acid encoding such a polypeptide. The cell can be either a prokaryotic or eukaryotic cell. In some embodiments, the nucleotide sequence is incorporated into a plasmid or viral vector capable of autonomous replication in the recipient cell. Any of a wide variety of vectors can be employed for this purpose and are known and available to those or ordinary skill in the art. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

In some embodiments, the composition a polypeptide as described herein or a nucleic acid encoding polypeptide as described herein can be a lyophilisate.

In some embodiments, the technology described herein relates to a syringe or catheter, comprising a therapeutically effective amount of a composition described herein.

In one aspect, described herein is a kit comprising a composition as described herein, e.g., a composition comprising a peptide as described herein. A kit is any manufacture (e.g., a package or container) comprising at least one reagent, e.g., a peptide, the manufacture being promoted, distributed, or sold as a unit for performing the methods described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to treat a subject with inflammation. Still in accordance with the present invention, “instructions for use” may include a tangible expression describing the preparation of at least one reagent described herein, such as dilution, mixing, or dosing instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of at least one reagent described herein. The packaging material generally has an external label which indicates the contents and/or purpose of the kit and/or its components.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g., inflammation or infection. A subject can be male or female.

A subject can be one who has been previously diagnosed with or identified as suffering from or having a condition in need of treatment (e.g. inflammation or infection) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect of the full length polypeptide. Conservative substitution variants that maintain the activity of wildtype will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of inflammation or infection as described herein.

In some embodiments, a polypeptide can be a variant of a sequence described herein, e.g. a variant of a polypeptide described herein. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of the wildtype, e.g. 110%, 125%, 150%, 175%, 200%, 500%, 1000% or more.

One method of identifying amino acid residues which can be substituted is to align, for example, human to a homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp or BLASTn (available on the world wide web at blast.ncbi.nlm.nih.gov), with default parameters set.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Typically conservative substitutions for one another also include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide as described herein can comprise at least one peptide bond replacement. A polypeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H). In some embodiments, a polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, can be modified, e.g. by addition of a moiety to one or more of the amino acids that together comprise the peptide. In some embodiments, a polypeptide as described herein can comprise one or more moiety molecules, e.g. 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG, albumin, or other fusion partners (e.g. Fc fragment of an immunoglobin).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established. Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having inflammation or infection, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the treatment described herein is exogenous. In some embodiments of any of the aspects, the treatment described herein is ectopic. In some embodiments of any of the aspects, the treatment described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to one or more signs or symptoms of inflammation or infection, e.g., neutrophil migration or invasion. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean #1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149×); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338×, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, a epidermal skin sampling technique, and/or collection of a secreted or expelled bodily fluid (e.g., mucus, urine, sweat, etc), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A method of treating inflammation in a subject in need thereof, the method comprising administering an anti-inflammatory or anti-infective treatment to a subject with an increased level of fragmented VEGF (fVEGF).
2. The method of paragraph 1, wherein the subject is a subject determined to have an increased level of fragmented VEGF (fVEGF).
3. The method of paragraph 1, further comprising a first step of detecting or measuring the level of fragmented VEGF (fVEGF) in the subject or a sample obtained from the subject.
4. The method of any one of the preceding paragraphs, wherein the anti-inflammatory reduces the rate of formation of fVEGF.
5. The method of any one of the preceding paragraphs, wherein the anti-inflammatory is PR1P.
6. A method of treating inflammation or infection in a subject in need thereof, the method comprising administering PR1P to the subject.
7. The method of any one of the preceding paragraphs, wherein the fVEGF is VEGF₁₆₅.
8. The method of any one of the preceding paragraphs, wherein the level of fVEGF is the level of fVEGF in the blood of the subject, or a blood sample of the subject.
9. The method of any one of the preceding paragraphs, wherein the level of fVEGF is the level of fVEGF in the injured or infected tissue of the subject, or an injured or infected tissue sample of the subject.
10. The method of any one of the preceding paragraphs, wherein the level of fVEGF is the local level of fVEGF.
11. The method of any one of the preceding paragraphs, wherein the level of fVEGF is not the systemic level of fVEGF.
12. The method of any one of the preceding paragraphs, wherein the level of fVEGF is the level of fVEGF at a site of injury and/or infection.
13. The method of any one of the preceding paragraphs, wherein the level of fVEGF is the level of fVEGF in the blood, serum, plasma, eye humor, saliva, urine, bronchoalveolar lavage fluid, cerebrospinal fluid, or exhaled breath condensate of the subject; or a blood, serum, plasma, eye humor, saliva, urine, bronchoalveolar lavage fluid, cerebrospinal fluid, or exhaled breath condensate sample of the subject.
14. The method of any one of the preceding paragraphs, wherein the fVEGF comprises, consists of, or consists essentially of:

(SEQ ID NO: 12)
ARQENPCGPCSERRKHLVQDPQTCKCSCKNTDSRCKARQLELNERTCRC
DKPR.

In some embodiments, the present technology may be defined in any of the following numbered paragraphs:
1. A peptide comprising the sequence of SEQ ID NO: 1 (DRVQRQTTTVVA).
2. The peptide of paragraph 1, consisting of the sequence of SEQ ID NO: 1.
3. A pharmaceutical composition comprising the peptide of any of the preceding paragraphs and a pharmaceutically acceptable excipient.
4. A method of treating inflammation in a subject in need thereof, the method comprising administering an anti-inflammatory or anti-infective treatment to a subject with an increased level of fragmented VEGF (fVEGF).
5. The method of paragraph 4, wherein the subject is a subject determined to have an increased level of fragmented VEGF (fVEGF).
6. The method of paragraph 4, further comprising a first step of detecting or measuring the level of fragmented VEGF (fVEGF) in the subject or a sample obtained from the subject.
7. The method of any one of paragraphs 4-6, wherein the anti-inflammatory reduces the rate of formation of fVEGF.
8. The method of any one of paragraphs 4-7, wherein the anti-inflammatory is the peptide or pharmaceutical composition of any one of paragraphs 1-3.
9. A method of treating inflammation or infection in a subject in need thereof, the method comprising administering the peptide or pharmaceutical composition of any one of paragraphs 1-3 to the subject.
10. The method of any one of paragraph 4-9, wherein the fVEGF is VEGF₁₆₅.
11. The method of any one of paragraph 4-10, wherein the level of fVEGF is the level of fVEGF in the blood of the subject, or a blood sample of the subject.
12. The method of any one of paragraph 4-11, wherein the level of fVEGF is the level of fVEGF in the injured or infected tissue of the subject, or an injured or infected tissue sample of the subject.
13. The method of any one of paragraph 4-12, wherein the level of fVEGF is the local level of fVEGF.
14. The method of any one of paragraph 4-13, wherein the level of fVEGF is not the systemic level of fVEGF.
15. The method of any one of paragraph 4-14, wherein the level of fVEGF is the level of fVEGF at a site of injury and/or infection.
16. The method of any one of paragraph 4-15, wherein the level of fVEGF is the level of fVEGF in the blood, serum, plasma, eye humor, saliva, urine, bronchoalveolar lavage fluid, cerebrospinal fluid, or exhaled breath condensate of the subject; or a blood, serum, plasma, eye humor, saliva, urine, bronchoalveolar lavage fluid, cerebrospinal fluid, or exhaled breath condensate sample of the subject.
17. The method of any one of paragraph 4-16, wherein the fVEGF comprises, consists of, or consists essentially of:

(SEQ ID NO: 2)
ARQENPCGPCSERRKHLVQDPQTCKCSCKNTDSRCKARQLELNERTCRC
DKPRR.

18. The peptide or composition of any one of paragraphs 1-3 for use in a method of treating inflammation.
19. An anti-inflammatory or anti-infective for use in a method of treating inflammation in a subject with an increased level of fragmented VEGF (fVEGF).
20. The peptide, composition, anti-inflammatory, or anti-infective of any one of paragraphs 21-22, wherein the subject is a subject determined to have an increased level of fragmented VEGF (fVEGF).
21. The peptide, composition, anti-inflammatory, or anti-infective of any one of paragraphs 21-23, wherein the anti-inflammatory reduces the rate of formation of fVEGF.
22. The peptide, composition, anti-inflammatory, or anti-infective of any one of paragraphs 21-24, wherein the fVEGF is VEGF₁₆₅.
23. The peptide, composition, anti-inflammatory, or anti-infective of any one of paragraphs 21-25, wherein the level of fVEGF is the level of fVEGF in the blood of the subject, or a blood sample of the subject.
24. The peptide, composition, anti-inflammatory, or anti-infective of any one of paragraphs 21-26, wherein the level of fVEGF is the level of fVEGF in the injured or infected tissue of the subject, or an injured or infected tissue sample of the subject.
25. The peptide, composition, anti-inflammatory, or anti-infective of any one of paragraphs 21-27, wherein the level of fVEGF is the local level of fVEGF.
26. The peptide, composition, anti-inflammatory, or anti-infective of any one of paragraphs 21-28, wherein the level of fVEGF is not the systemic level of fVEGF.
27. The peptide, composition, anti-inflammatory, or anti-infective of any one of paragraphs 21-29, wherein the level of fVEGF is the level of fVEGF at a site of injury and/or infection.
28. The peptide, composition, anti-inflammatory, or anti-infective of any one of paragraphs 21-30, wherein the level of fVEGF is the level of fVEGF in the blood, serum, plasma, eye humor, saliva, urine, bronchoalveolar lavage fluid, cerebrospinal fluid, or exhaled breath condensate of the subject; or a blood, serum, plasma, eye humor, saliva, urine, bronchoalveolar lavage fluid, cerebrospinal fluid, or exhaled breath condensate sample of the subject.
29. The peptide, composition, anti-inflammatory, or anti-infective of any one of paragraphs 21-31, wherein the fVEGF comprises, consists of, or consists essentially of:

(SEQ ID NO: 2)
ARQENPCGPCSERRKHLVQDPQTCKCSCKNTDSRCKARQLELNERTCRC
DKPRR.

The technology described herein is further illustrated by the provided examples which in no way should be construed as being further limiting.

### EXAMPLES

### Example 1

Vascular Endothelial Growth Factor (VEGF) is a family of growth factors that regulate a wide range of biological and functional signaling pathways4 that are important in the maintenance of cells and tissue health and in the process of healing following tissue injury4,5. Its role in inflammation is not well understood. Degradation of VEGF by proteases (enzymes which bind and degrade proteins) such as elastase and plasmin that are present in normal tissues but also released into tissues by inflammatory cells at sites of tissue injury and during early inflammation leads to the generation of smaller VEGF fragments (fVEGF) that have altered signaling properties compared to the parent VEGF molecules6-8. The significance of fVEGF and its role in VEGF signaling and in inflammation is neither well characterized nor understood. It is hypothesized that fVEGF levels are negligible in healthy tissues (where they are produced by baseline levels of VEGF proteases during the natural life cycle of VEGF production and degradation) and are elevated in injured/inflamed tissues where protease levels are high. Kurtagic et al found that fVEGF is chemoattractant to macrophages (a type of inflammatory cell that ingests and removes foreign particles (dead or alive) from injured/infected tissues and cells).

The inventors have discovered that:
1) fVEGF is chemoattractant to human neutrophils, which are the earliest inflammatory cells that appear at sites of organ/tissue injury or following infection and which initiate the inflammatory/immune response.
2) PR1P, a novel peptide, which binds VEGF and protects VEGF from degradation by proteases such as elastase and plasmin, prevents the generation of fVEGF
3) PR1P prevents neutrophil migration into injured tissues and reduces inflammation in multiple animal tissue injury models

It is therefore contemplated that fVEGF represents a previously unrecognized pivotal signaling molecule which mediates inflammation, and whose presence within tissues and fluids indicates active inflammation. It is further contemplated that fVEGF levels in tissues or fluids can be used to diagnose inflammation and to guide therapy directed at reducing inflammation or eradicating infection (note: active infection results in ongoing inflammation).

Described herein is:
1) a biologically active fragment of VEGF (fVEGF), and
2) the use of (fVEGF) as a diagnostic tool to gauge the degree of inflammation in a specific organ or within tissue or fluid from an organ, or in the whole body in general.
3) A treatment to reduce inflammation, peptides (as PR1P), small molecules, by blocking VEGF's protease cleavage sites from producing fVEGF.

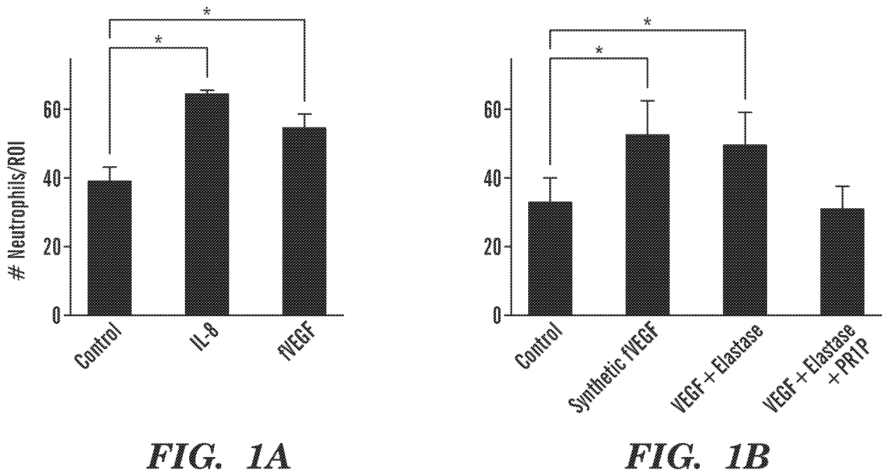

As noted above fVEGF was recently shown to be a chemoattractant to macrophages 8 and so the inventors hypothesized that it similarly mediates neutrophil migration. This is important because neutrophils are the first inflammatory cells that arrive in tissues that are injured, from most any cause: the arrival of neutrophils is due to their attraction to chemicals or products released by cells during injury. This first step is an important process in enabling the immune system to mount an appropriate response to injury or an invading organism, and begins the process of “healing” (e.g., recruitment of additional immune cells to either fight an infection or restore tissue to health). Importantly, the inventors recently showed that PR1P, a short (12 amino acid) peptide they designed protects VEGF from protease degradation by binding to VEGF and preventing the formation of fVEGF. To test whether fVEGF mediates neutrophil migration in vitro and whether PR1P mediates neutrophil migration via inhibiting the formation of fVEGF, the inventors modified an inflammatory cell migration assay 1 to test the effect of commercially synthesized fVEGF, and of fVEGF formed via proteolytic degradation of VEGF in the presence and absence of PR1P on human neutrophil migration. As shown in FIGS. 1A and 1B, IL-8 (a common cytokine and known neutrophil chemoattractant), commercially synthesized fVEGF, and the product of VEGF pre-incubated with neutrophil derived elastase, induced migration of human neutrophils. In contrast, when PR1P was present during elastase treatment of VEGF, the ensuing product, which lacks fVEGF, failed to induce neutrophil migration, indicating that PR1P prevents the generation of fVEGF and subsequent induction of neutrophil migration (FIG. 1B). Thus, fVEGF is a chemoattractant to neutrophils in vitro.

PR1P, which prevents VEGF degradation and the production of fVEGF by proteases, reduces neutrophil migration into, and inflammation within injured tissues in vivo.

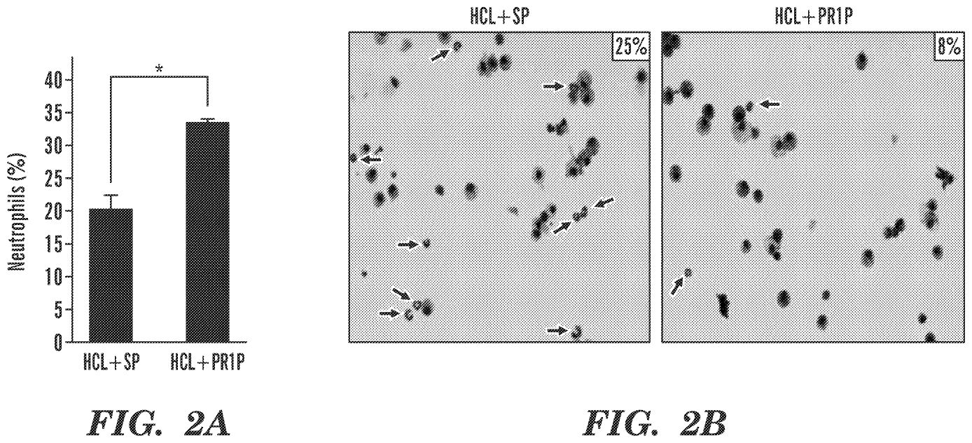

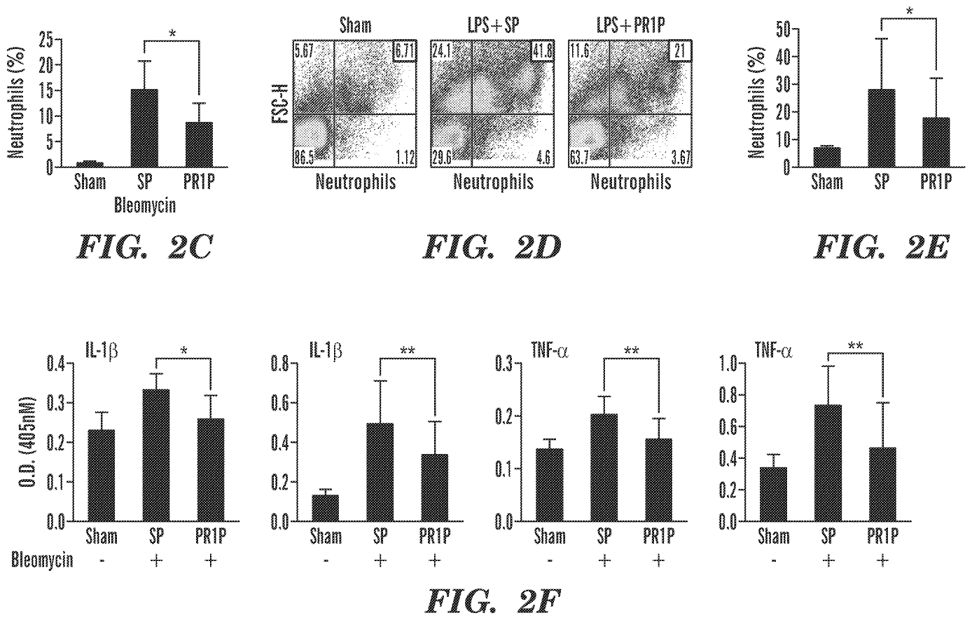

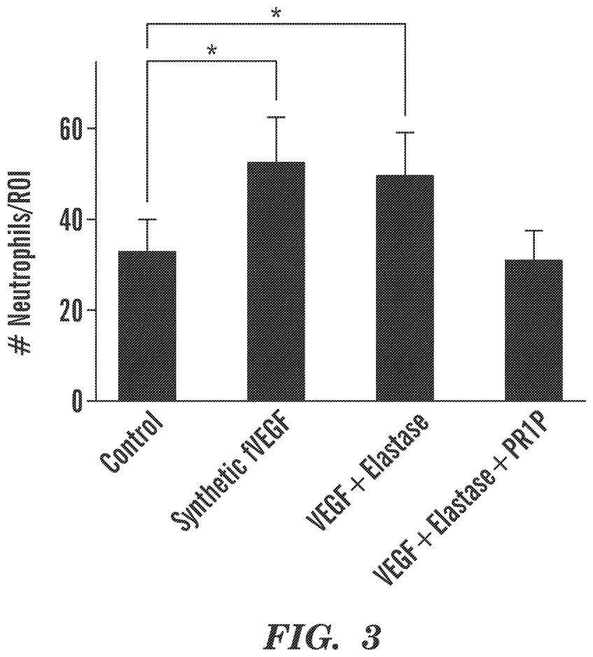

Circulating neutrophils are often the first immune cells to arrive at sites of tissue injury and are thought to initiate the necessary inflammatory response required to restore tissue integrity (9). The inventors therefore sought to determine whether PR1P could be used to reduce neutrophil migration into injured lungs and/or GI tract following tissue injury in established animal models. In so doing, the inventors assumed to indirectly test the hypothesis that fVEGF mediated neutrophil chemoattraction in vivo and inflammation in particular. Lung injury models were chosen to characterize the effect of PR1P on inflammation from diverse insults and injury mechanisms, and over different time courses. Specifically, lung injury was induced either by inhalation of lipopolysaccharides (LPS, a component of the bacterial cell wall and used as a potent trigger so acute lung inflammation whereby neutrophils migrate into the lungs within 4-24 hours (10)) or by intra-tracheal administration of bleomycin (11) which induces acute inflammation and later fibrosis through a multi-factorial mechanism, or HCL (12) (also multifactorial in nature through damage to the alveolar-capillary membrane (13), resulting in immediate polymorphonuclear neutrophil (PMN) recruitment (14), pulmonary edema, and hypoxia (15)). In each of the lung injury models, tissue injury was following by daily inhalation of PR1P or scrambled peptide (SP) until completion of each of the experiments according to the injury model (ie., LPS and HCL for 24 hours and bleomycin for 72 hours). Fluoresecnt Antibody Cell Sorting (FACS) analyses of BALF following HCL- and bleomycin-induced lung injury reveal that inhaled PR1P significantly reduced toxin-induced neutrophil migration into the airways (i.e., BALF, HCL FIG. 2A; Bleomycin FIG. 2C). Representative DIFF-QUIK stained cells used in the FACS analyses following acid injury are shown in FIG. 2B. Similarly, FACS analyses of cells from whole lung digests from LPS injured lungs show that inhaled PR1P significantly reduced the presence of migrated neutrophils into the lungs 24 h after exposure to LPS (FIGS. 2D-2E).

To determine whether reduced neutrophil migration into lungs correlated with reduced inflammation, the inventors measures the levels of pro-inflammatory cytokines in BALF following bleomycin injury and found that inhaled PR1P significantly reduced levels of TNF-α, IL-1β, and IL-6 compared to scrambled peptide (FIG. 2F). Furthermore, inhaled PR1P reduced myeloperoxidase (MPO) activity in lung tissue following bleomycin injury (FIG. 2F). Together these data suggest that inhaled PR1P effectively reduces neutrophil migration into the lungs, and in so doing PR1P attenuates lung inflammation as determined by reduced cytokine levels and MPO activity.

It is contemplated herein that fVEGF, derived through VEGF degradation, mediates inflammation (chemoattracts neutrophils which initiate and mediate inflammation). It is further contemplated herein that the measurement of fVEGF could be used as a biomarker of inflammation. It is contemplated herein that fVEGF may serve as a point of convergence in VEGF signaling that determines whether tissues being to heal and remodel or remain inflamed. Thus, the quantification of fVEGF in tissues or fluids could serve as a diagnostic tool to gauge the degree of inflammation in specific organs (or parts of organs) or in the whole body in general.

### REFERENCES

1 Kurtagic, E., Jedrychowski, M. P. & Nugent, M. A. Neutrophil elastase cleaves VEGF to generate a VEGF fragment with altered activity. Am J Physiol Lung Cell Mol Physiol 296, L534-546, doi: 10.1152/ajplung.90505.2008 (2009).
2 Pease, J. E. & Sabroe, I. The role of interleukin-8 and its receptors in inflammatory lung disease: implications for therapy. Am J Respir Med 1, 19-25, doi: 10.1007/BF03257159 (2002).
3 fda.gov/media/71278/download.
4 Ferrara, N. Vascular endothelial growth factor: basic science and clinical progress. Endocr Rev 25, 581-611, doi: 10.1210/er.2003-0027 (2004).
5 Senger, D. R. et al. Stimulation of endothelial cell migration by vascular permeability factor/vascular endothelial growth factor through cooperative mechanisms involving the alphavbeta3 integrin, osteopontin, and thrombin. Am J Pathol 149, 293-305 (1996).
6 Lauer, G. et al. Expression and proteolysis of vascular endothelial growth factor is increased in chronic wounds. The Journal of investigative dermatology 115, 12-18, doi: 10.1046/j. 1523-1747.2000.00036.x (2000).
7 Lauer, G., Sollberg, S., Cole, M., Krieg, T. & Eming, S. A. Generation of a novel proteolysis resistant vascular endothelial growth factor165 variant by a site-directed mutation at the plasmin sensitive cleavage site. FEBS Lett 531, 309-313, doi: 10.1016/s0014-5793 (02) 03545-7 (2002).
8 Kurtagic, E., Rich, C. B., Buczek-Thomas, J. A. & Nugent, M. A. Neutrophil Elastase-Generated Fragment of Vascular Endothelial Growth Factor-A Stimulates Macrophage and Endothelial Progenitor Cell Migration. PLOS One 10, e0145115, doi: 10.1371/journal.pone.0145115 (2015).
9 Selders, G. S., Fetz, A. E., Radic, M. Z. & Bowlin, G. L. An overview of the role of neutrophils in innate immunity, inflammation and host-biomaterial integration. Regen Biomater 4, 55-68, doi: 10.1093/rb/rbw041 (2017).
10 Chen, H., Bai, C. & Wang, X. The value of the lipopolysaccharide-induced acute lung injury model in respiratory medicine. Expert Rev Respir Med 4, 773-783, doi: 10.1586/ers. 10.71 (2010).
11 Ge, V., Banakh, I., Tiruvoipati, R. & Haji, K. Bleomycin-induced pulmonary toxicity and treatment with infliximab: A case report. Clin Case Rep 6, 2011-2014, doi: 10.1002/ccr3.1790 (2018).
12 Zarbock, A., Singbartl, K. & Ley, K. Complete reversal of acid-induced acute lung injury by blocking of platelet-neutrophil aggregation. J Clin Invest 116, 3211-3219, doi: 10.1172/JCI29499 (2006).
13 Eijking, E. P., Gommers, D., So, K. L., Vergeer, M. & Lachmann, B. Surfactant treatment of respiratory failure induced by hydrochloric acid aspiration in rats. Anesthesiology 78, 1145-1151, doi: 10.1097/00000542-199306000-00019 (1993).
14 Goldman, G. et al. Reactive oxygen species and elastase mediate lung permeability after acid aspiration. J Appl Physiol (1985) 73, 571-575, doi: 10.1152/jappl. 1992.73.2.571 (1992). 15 Nagase, T. et al. Acute lung injury by sepsis and acid aspiration: a key role for cytosolic phospholipase
A2. Nat Immunol 1, 42-46, doi: 10.1038/76897 (2000).

### Example 2

The invention provides a rapid biomarker of inflammation. The general purpose of the invention is to rapidly diagnose or measure the degree of inflammation during illness. In addition, the invention allows clinicians to gauge the success of anti-inflammatory or anti-bacterial (or viral) treatments in real time.

VEGF is ubiquitous, expressed in all organs of the body, and so VEGF fragments could be similarly detected in all human tissues and fluids including in the blood, serum, plasma, eye (humor), saliva, urine, Broncho Alveolar Lavage Fluid (BALF), Cerebrospinal fluid (CSF), and even in

Exhaled Breath Condensate (EBC). Existing methods capable of identifying and quantifying presence or relative levels of fVEGF include Western Blot technologies for fluids with high or moderate concentrations of fVEGF. Thus, due to the ubiquitous nature of fVEGF, the primary applications for this invention are any inflammatory disease (of any cause) involving any organ of the body.

fVEGF is a naturally occurring protein degradation product of the original VEGF parent molecule. fVEGF is generated in all tissues of the body when proteases such as elastase or plasmin that are present in low concentrations normally in tissues, or when deposited into tissues by inflammatory cells during inflammation degrade VEGF. As noted above, fVEGF displays altered receptor binding and signaling properties compared to the parent VEGF molecule. Mass spectrometry analysis of fVEGF suggests that proteases may cleave VEGF at or within the amino-(N—) and carboxy (C—) termini as well as within internal regions of VEGF leading to degradation products with altered structural motifs thus altering binding and signaling properties. One fVEGF amino acid sequence is ARQENPCGPCSERRKHLVQDPQTCKCSCKNTDSRCKARQLELNERTCRCDKPRR

(SEQ ID NO: 2). Western blot analysis can detect fVEGF using antibodies against the parent VEGF molecule. It is contemplated that fVEGF can be detected in nanomolar concentration in ul samples of body fluids (i.e. to detect fVEGF in blood, serum, plasma, eye humor, saliva, urine, bronchoalveolar lavage fluid, cerebrospinal fluid, and exhaled breath condensate).

Many existing biomarker assays (e.g., white blood cell count, C-Reactive Protein (CRP), the erythrocyte sedimentation rate (ESR), procalcitonin, serum amyloid A, cytokines, alpha-1-acid glycoprotein, plasma viscosity, ceruloplasmin, hepcidin, and haptoglobin) are disease specific and/or may take several hours to days to result and require expensive, less available and specialized techniques and assays to deliver results. As a result, many of these tests fail to deliver useful information within the demanded timeframe to assist clinical decision making in real time. As a result of the direct correlation between fVEGF and inflammation in general, the present technology provides an affordable, non-specific and rapid assay to measure fVEGF concentration and assess the degree of inflammation in the whole body (i.e. blood, serum) or in organ specific fluids.

### REFERENCES

Kurtagic, E., Jedrychowski, M. P. & Nugent, M. A. Neutrophil elastase cleaves VEGF to generate a VEGF fragment with altered activity. Am J Physiol Lung Cell Mol Physiol 296, L534-546, doi: 10.1152/ajplung.90505.2008 (2009).
Lauer, G. et al. Expression and proteolysis of vascular endothelial growth factor is increased in chronic wounds. The Journal of investigative dermatology 115, 12-18, doi: 10.1046/j.1523-1747.2000.00036.x (2000).
Kurtagic, E., Rich, C. B., Buczek-Thomas, J. A. & Nugent, M. A. Neutrophil Elastase-Generated Fragment of Vascular Endothelial Growth Factor-A Stimulates Macrophage and Endothelial Progenitor Cell Migration. PLOS One 10, e0145115, doi: 10.1371/journal.pone.0145115 (2015).

### Example 3

PR1P, a VEGF-stabilizing peptide, reduces injury and inflammation in acute lung injury and ulcerative colitis animal models

Acute Respiratory Distress Syndrome (ARDS) and Ulcerative Colitis (UC) are each characterized by tissue damage and uncontrolled inflammation. Neutrophils and other inflammatory cells play a primary role in disease progression by acutely responding to direct and indirect insults to tissue injury and by promoting inflammation through secretion of inflammatory cytokines and proteases. Vascular Endothelial Growth Factor (VEGF) is a ubiquitous signaling molecule that plays a key role in maintaining and promoting cell and tissue health, and is dysregulated in both ARDS and UC. Recent evidence suggests a role for VEGF in mediating inflammation, however, the molecular mechanism by which this occurs is not well understood. We recently showed that PR1P, a 12-amino acid peptide that binds to and upregulates VEGF, stabilizes VEGF from degradation by inflammatory proteases such as elastase and plasmin thereby limiting the production of VEGF degradation products (fragmented VEGF (fVEGF)). Here we show that fVEGF is a neutrophil chemoattractant in vitro and that PR1P can be used to reduce neutrophil migration in vitro by preventing the production of fVEGF during VEGF proteolysis. In addition, inhaled PR1P reduced neutrophil migration into airways following injury in three separate murine acute lung injury models including from lipopolysaccharide (LPS), bleomycin and acid. Reduced presence of neutrophils in the airways was associated with decreased pro-inflammatory cytokines (including TNF-α, IL-1β, IL-6) and Myeloperoxidase (MPO) in broncho-alveolar lavage fluid (BALF). Finally, PR1P prevented weight loss and tissue injury and reduced plasma levels of key inflammatory cytokines IL-1β and IL-6 in a rat TNBS-induced colitis model. Taken together, our data demonstrate that VEGF and fVEGF may each play separate and pivotal roles in mediating inflammation in ARDS and UC, and that PR1P, by preventing proteolytic degradation of VEGF and the production of fVEGF may represent a novel therapeutic approach to preserve VEGF signaling and inhibit inflammation in acute and chronic inflammatory diseases.

### INTRODUCTION

ARDS and UC are two common diseases characterized by tissue injury, VEGF dysregulation and severe inflammation. ARDS is an acute and fulminant form of respiratory failure characterized by lung inflammation, hypoxemia and multi-organ failure requiring mechanical ventilation and prolonged ICU hospitalization (1, 2). There are no medical therapies to prevent or cure ARDS and management of ARDS remains supportive (3). UC is similarly characterized by chronic inflammation within the gastro-intestinal tract, and is associated with weight loss, abdominal pain, recurrent diarrhea, and bleeding (4). Treatments for UC include immunomodulatory medicines that are often insufficient to control disease. Proctocolectomy surgery remains the only curative option and is utilized in 15% of all UC patients (5). Neutrophils are the most abundant white blood cells in humans and are the first circulating inflammatory cell to arrive at sites of tissue injury during acute inflammation such as seen in ARDS and UC (6). Upon neutrophil activation, neutrophil-derived proteases including elastase, plasmin, MMP-8, MMP-9, and pro-inflammatory cytokines, including tumor necrosis factor α (TNF-α), interleukin (IL)-1β, and IL-6, are released at the sites of inflammation leading to structural local tissue damage and amplification of the inflammatory response. Further recruitment of neutrophils and other inflammatory cells including lymphocytes and macrophages during ARDS (7, 8) and UC (9, 10) ensues, which in turn contributes to heightened inflammation, the production of reactive oxygen species (ROS) (11) with disruption of cell membranes and ongoing tissue damage. Curiously, neutrophils and other inflammatory cells are also required to prevent invasion of disrupted epithelial cell barriers by infectious pathogens, and so anti-inflammation strategies envisioned to cure or treat ARDS and/or UC must balance their effects on inflammatory cell function and numbers. Also, as noted above, VEGF dysregulation is often associated with tissue injury and inflammation, and a clear understanding of how VEGF might simultaneously mediate inflammation and restoration of tissue to health is not clear.

VEGFs are a family of endogenous growth factors with angiogenic, survival and anti-apoptotic properties (12, 13). VEGF-A is a highly conserved member of the VEGF family, and VEGF165 (a 165-amino acid isoform of VEGF-A, henceforth referred to as VEGF) is an ubiquitous angiogenic and survival factor that binds VEGF receptor 1 (VEGFR1) and 2 (VEGFR2), as well as neuropilin-1 (NRP-1) (14). These receptors are on multiple cell types and tissues throughout the body including lung endothelial and epithelial cells, neutrophils and other inflammatory cells that are resident in, or which infiltrate the lungs and GI tract following injury (10, 15). VEGF signaling leads to a wide variety of biological and functional outcomes that are critical in maintenance of cell and tissue health and healing following tissue insults (16-19). Neutrophil derived proteases such as elastase and plasmin cleave VEGF (20-22) into a smaller VEGF isomer (fragmented VEGF (fVEGF)) with altered VEGF signaling (20), and so an important but underrecognized effect of increased neutrophil activity during acute and chronic inflammation such as seen in ARDS (23, 24) and UC (25) is VEGF dysregulation.

We recently developed a 12 amino-acid VEGF binding peptide (PR1P (26)) that protects VEGF from degradation by the proteases elastase and plasmin, and upregulates VEGF signaling (27). We and others have identified potential therapeutic efficacy for PR1P in multiple animal models all characterized by tissue injury with subsequent inflammation and VEGF dysregulation, including in emphysema (27), myocardial infarction (28), nerve degeneration (29), hind-limb and cerebral ischemia (26, 30), ligament regeneration (31), and chronic wounds (32). fVEGF, the by-product of VEGF degradation, was recently shown to be a chemoattractant to macrophages (20, 21). We therefore hypothesized that fVEGF is a chemoattractant to neutrophils, and that by preventing fVEGF production via VEGF stabilization, PR1P reduces neutrophil migration into sites of ongoing inflammation all the while upregulating VEGF signaling by preserving local VEGF levels and concentration gradients.

To test this hypothesis, we first designed an in vitro neutrophil migration assay to characterize the ability of PR1P to mediate fVEGF induced neutrophil chemoattraction, and then tested the potential of PR1P to mitigate inflammation in lung injury and ulcerative colitis animal models. Here we present data showing that fVEGF is chemoattractant to neutrophils in vitro, and that by blocking the production of fVEGF during VEGF proteolysis by elastase, PR1P can mediate VEGF dependent neutrophil chemoattraction. Subsequently, inhaled PR1P reduced neutrophil migration into the airways following injury in three separate murine acute lung injury models including from lipopolysaccharide (LPS), bleomycin and acid. Reduced presence of neutrophils in the airways was associated with decreased pro-inflammatory cytokines (including TNF-α, IL-1β, IL-6, and Myeloperoxiase (MPO)) in broncho-alveolar lavage fluid (BALF). Finally, PR1P prevented weight loss and tissue injury and reduced plasma levels of key inflammatory cytokines IL-1β and IL-6 in a TNBS-induced colitis model in the rat. Together these data demonstrate that fVEGF may play a key role in mediating inflammation in acute lung injury (ALI) and UC, and that PR1P may represent a novel therapeutic approach to stabilize VEGF and preserve VEGF signaling and inhibit inflammation to improve outcome in acute and chronic inflammatory diseases.

### Materials and Methods

### Mice

6-week-old female C57BL/6J mice were purchased from Jackson Laboratory (Bar Harbor, ME) and were kept in a pathogen-controlled environment in standard cages and were fed ad libitum. Protocols for the in vivo studies were approved by the Institutional Animal Care and Use Committee (IACUC) at Boston Children's Hospital.

### Rats

Experiments with rats were performed by the CRO Washington Biotechnology Inc. (Simpsonville, MD). Male Sprague-Dawley rats were purchased from Envigo (Dublin, VA). The rats were cared for per company protocol, were examined daily and found to be free of any clinical signs of disease or distress.

### Peptides

The 12-mer peptides PR1P (DRVQRQTTTVVA (SEQ ID NO: 1)), scrambled PR1P(VRQVVTARDTTQ (SEQ ID NO: 13)) and fVEGF (ARQENPCGPCSERRKHLVQDPQTCKCSCKNTDSRCKARQLELNERTCRCDK PRR (SEQ ID NO: 2)) were all synthetized by Genscript (Piscataway, NJ).

Induction of ulcerative colitis in rats by tri-nitrobenzene sulfonic acid

The tri-nitrobenzene sulfonic acid (TNBS) model is the most commonly used model to investigate the pathogenesis of, and for developing new anti-inflammatory strategies for ulcerative colitis (UC) (33). Colonic damage is created by the intrarectal application of a barrier disrupting ethanol and hapten mixture and since ethanol splits the epithelial layer, it allows the lamina propria to be exposed to bacterial components (34, 35). The rat model was performed by the (CRO) Washington Biotechnology (Simpsonville, MD). Sprague-Dawley male rats (210-230 grams) were randomly divided into five experimental groups with eight rats in each group including: (1) vehicle control, (2) TNBS-treated, (3) TNBS+PR1P (0.5 mg/kg), (4) TNBS+PR1P (5 mg/kg), and (5) TNBS+prednisolone, (50 mg/kg). Rats were deprived of food for 24 hours, anesthetized with intraperitoneal ketamine (80 mg/kg)+xylazine (10 mg/kg) and a polyurethane nutritional cannula was inserted through the anus. To induce acute colitis, 48 mg/kg TNBS dissolved in 0.25 mL 50% ethanol was instilled into the colon through the cannula. Control animals were instilled with vehicle (50% ethanol in saline). To evaluate the potential effects of PR1P in TNBS-induced colitis, rats were treated daily for 7 days with either PR1P, prednisolone or saline as noted above. Body weight, stool consistency and quality (diarrhea, blood) were recorded daily. Rats were sacrificed on day 7, weighed, and blood collected and stored using pre-chilled EDTA-microtainer tubes (Becton-Dickinson). Blood samples were processed for plasma and stored at −80° C. After a midline incision in the abdomen, the colon was scored for adhesions and stricture. The colon was then removed from the rectum to the ileo-caecal juncture and the colon length recorded. After a longitudinal incision of the colon, the fecal material was removed and the colon weight, colon wall thickness, ulcer length, and ulcer width were recorded. A Colonic Score (maximum=12) was calculated as follows: Adhesion: (i) none=0, ii) minimal=1, iii) involving several bowel loops=2, Stricture: i) none=0, ii) minimal=1, iii) mild=2, iv) severe, proximal dilatation=3, Ulcer: i) none=0, ii) focal hyperemia, no ulcers=1, iii) ulcer without significant inflammation (hyperemia and bowel wall thickening=2, iv) ulceration of 1-¾ 3 cm=3, v) ulceration>3-<6 cm=4, vi) ulceration≥6 cm=5, Wall thickness: i) less than 1 mm=0, ii) 1-3 mm=1, iii)>3 mm=2. A section of the colon was preserved in 20 volume 10% neutral buffered formalin (Richard-Allan Scientific). The carcasses were disposed of appropriately. The formalin-preserved colon samples were submitted for histopathological processing and evaluation. Each tissue was embedded longitudinally in its own block. One slide per block was sectioned at −4 μm and stained with hematoxylin and eosin (H&E). Slides were evaluated with light microscopy by an ACVP board-certified veterinary pathologist. Histologic findings were diagnosed and were given a severity score of 0-5 (0=not present/normal, 1=minimal, 2=mild, 3=moderate, 4=marked, 5=severe). Features were scored according to extent as follows: Grade 0: Not present/within normal limits, Grade 1: Minimal; <10% of sample affected, Grade 2: Mild; 10-25% of sample affected, Grade 3: Moderate; 26-50% of sample affected, Grade 4: Marked; 51-75% of tissue affected, Grade 5: Severe; >75% of tissue affected.

### Murine LPS Model of Acute Lung Injury

Mice (22+2 g) were placed in a whole-animal nebulization chamber (14×5×8 cm) and allowed to spontaneously breathe nebulized lipopolysaccharide LPS (Salmonella enterica) (Sigma-Aldrich, St. Louis, MO) (0.6 mg/mice), followed by PR1P or scrambled peptide (SP) (300 μg/ml in 3 ml of 0.9% normal saline) for 20 minutes (Proneb Ultra II Nebulizer, Pari Respiratory Equipment). Animals were sacrificed 24 h after inhalation experiments and lungs were harvested. The lung tissue was minced using surgical scissors and suspended in 3 ml of serum-free bronchial epithelial-cell growth basal medium containing Liberase (2 mg/ml; Sigma-Aldrich), Dispase (5 U/ml; Stemcell Technology), and DNase (50 U/ml; Sigma-Aldrich) for 30 minutes at 37° C. Digested tissue was filtered (70-mm mesh Thermo Fisher Scientific), and filtered cells were washed twice with FACS media (PBS containing 3% FCS) and centrifuged at 1,000 rotations/min for 5 minutes. Pelleted material was incubated for 90 seconds in erythrocyte-lysis buffer (Sigma-Aldrich), resuspended in FACS media, and prepared for FACS analysis.

### Urine Bleomycin Model of ALI

Mice were anesthetized with ketamine/xylazine (ketamine (125 mg/kg; Sanofi Winthrop Pharmaceuticals, New York, NY), xylazine (12.5 mg/kg; Phoenix Scientific, St. Joseph, MO) and injected intratracheally using a 24-gauge angiocatheter with 100 ul sterile PBS or bleomycin (Cayman Chemicals, Arbor, MI, 0.75 mg/kg dissolved in PBS). Four days later, animals were sacrificed and bronchoalveolar lavage fluid (BALF) was collected by instilling 1 mL chilled PBS into the lungs via a 24-gauge angiocatheter (Becton Dickinson) and collected. The procedure was repeated 5× each with fresh PBS. The BALF was then centrifuged for 5 min at 300 g. as analyzed for total cell count and leukocyte differential. Cells were characterized morphologically and counted by adhering them to glass slides using the Cytospin system (Shandon, Southern Sewickley, PA) and then staining them by Kwik-Diff (Fisher Scientific, Boston MA). The adhered stained cells were air-dried and counted in a blinded manner under a light microscope.

### Murine HCL Model of ALI

Mice were anesthetized with ketamine/xylazine (ketamine (125 mg/kg; Sanofi Winthrop Pharmaceuticals, New York, NY), xylazine (12.5 mg/kg; Phoenix Scientific, St. Joseph, MO) and 50 μl of hydrochloric acid (0.1 N HCl, pH 1.5, endotoxin free; Sigma-Aldrich) was instilled selectively into the left mainstem bronchus of anesthetized mice via a 24-gauge angiocatheter inserted intratracheally. 24 hours after acid instillation, bronchoalveolar lavage (BAL) was performed as described above and the BALF analyzed for total cell count and leukocyte differential as described above.

FACS and cytospin analysis of neutrophils derived from bronchoalveolar lavage fluid following HCL or bleomycin treatment

Three days after bleomycin and 24 h after HCL and LPS exposure, mice were anesthetized with an intraperitoneal injection of ketamine (125 mg/kg; Sanofi Winthrop Pharmaceuticals, New York, NY) and xylazine (12.5 mg/kg; Phoenix Scientific, St. Joseph, MO). The trachea was cannulated using a (24 gauge angiocatheter (Becton Dickinson), and BALF collected as described above. Filtered BALF and red blood cell-lysed blood cells were resuspended in FACS buffer (Mouse PBS, 2% fetal calf serum, 2 mM EDTA). Cells were stained with the two monoclonal specific monoclonal antibodies against neutrophils Anti-CD11B-PE and Ly6G-FITC (BD Bioscience, Woburn, MA) (20 min, 4° C. on ice). Cells were then washed and resuspended in 50 μl FACS buffer. All studies were performed on a FACS Calibur (Becton Dickinson, San Jose, CA), and data were analyzed with FlowJo software (Tree Star, Ashland, OR). To confirm the presence of neutrophils within the different flow cytometry samples, we separately characterized a sample from each group by adhering the cells to glass slides using the Cytospin system (Shandon, Southern Sewickley, PA) and then staining them by Kwik-Diff (Fisher Scientific, Boston MA). The adhered stained cells were air-dried and counted in a blinded manner under a light microscope.

### Cytokine Analysis

Murine BALF The Broncho-Alveolar Lavage fluid (BALF) supernatant from mice was collected following centrifugation (4 min at 7000 g) and stored at −80° C. Inflammatory cytokine levels (IL1-β, IL-6 and TNF-α) in the BALF were determined as per manufacturer instructions using Enzyme Linked Immunosorbent Assay (ELISA) kits obtained from Biolegend, Inc, (San Diego, CA).

Rat plasma Rats were anesthetized and bled into pre-chilled EDTA-microtainer tubes (Becton-Dickinson). The blood samples were processed to plasma which was stored in labeled 1.5-ml Eppendorf tubes at −80° C. Plasma samples were thawed to room temperature and assayed by ELISA for IL-6, IL-β and TNF-α (R&D Systems).

### Neutrophil Migration Assay

Neutrophil migration assays were performed using a modification of the Boyden chamber technique (20) using 24-well Transwell permeable supports (Corning, NY) with migration inserts (3.5 μm pore size, 6.5 mm diameter). The bottom parts of the 24-well plate were pre-incubated for 60 minutes in serum-free DMEM containing 25 mM HEPES, pH 7.5 at 37° C. (5% CO2, humidified) with either fVEGF (50 nM, Genscript (Piscataway, NJ)), elastase (100 μg/ml, Fitzgerald, UK)+VEGF (10 ng/ml, Peprotech, Israel), or elastase 1 μg/ml μg/ml+VEGF (10 vg/ml)+PR1P (100 μg/ml). At the end of the 60-minute (digestion period) human neutrophils (IQ Biosciences, Alameda, CA) were plated onto the upper Transwell inserts at a density of 100,000 cells/insert and allowed to migrate towards the bottom chamber for two hours (migration period). Upon conclusion of the migration period cells that had migrated through the membrane were scraped and collected and washed once in PBS without Ca2+ and Mg2+, fixed with 100% pre-chilled methanol for 10 min, adhered to glass coverslips and then stained by H&E. Images of migrated cells were captured by light microscopy from six random regions of interest (10× magnification) and counted.

### Results

PR1P mediates neutrophil migration in vitro by preventing VEGF degradation fVEGF was recently shown to be chemoattractant to macrophages (21) and so we hypothesized that it similarly mediates neutrophil migration. Furthermore, we recently showed that PR1P protects VEGF from neutrophil-derived protease degradation by binding to the heparin binding domain (HBD) (27) on VEGF and preventing the formation of fVEGF. We therefore surmised that by binding to and stabilizing VEGF from proteolytic degradation into fVEGF, PR1P indirectly mediates neutrophil chemoattraction. To test this hypothesis, we modified an inflammatory cell migration assay (20) to test the effect of fVEGF and PR1P on neutrophil migration. As shown in FIG. 3, both commercially synthesized fVEGF, as well as the product of VEGF pre-incubated with neutrophil derived elastase, induced migration of human neutrophils. In contrast, when PR1P was present during elastase treatment of VEGF, the ensuing product failed to induce neutrophil migration, indicating that PR1P prevents the generation of fVEGF and subsequent induction of neutrophil migration. Thus, PR1P indirectly influences neutrophil migration through its ability to protect VEGF from proteolytic degradation.

PR1P limits neutrophil migration to sites of tissue injury in lung and gastro-intestinal injury models in vivo

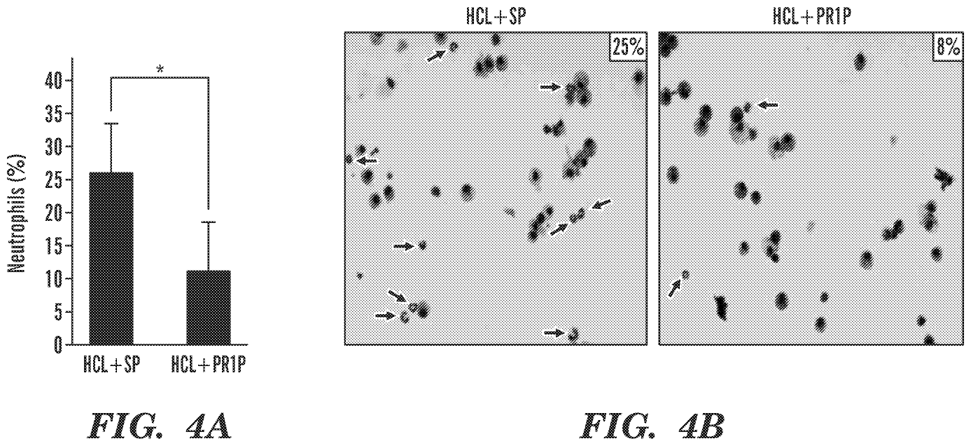

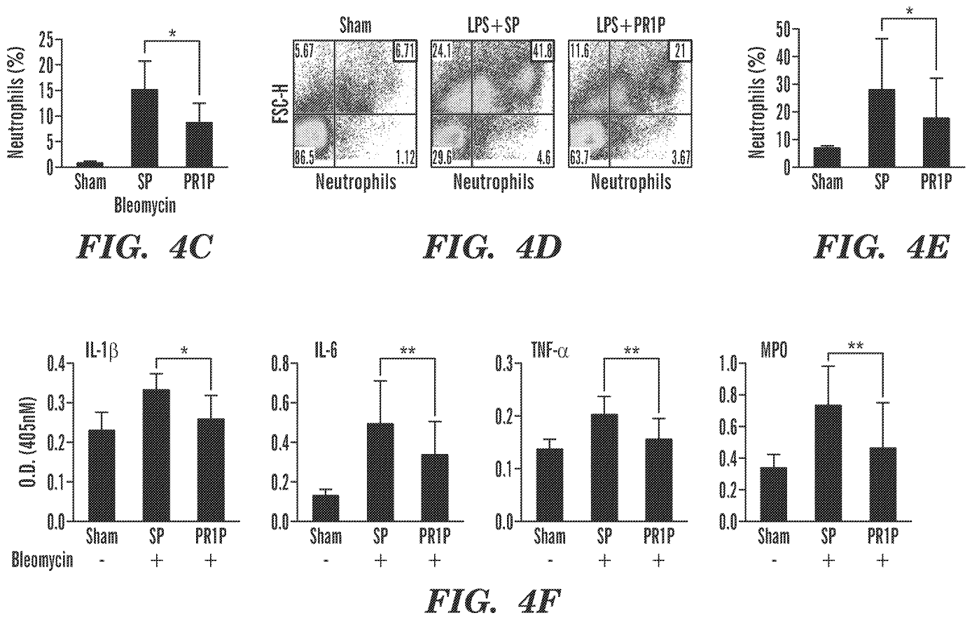

Circulating neutrophils are often the first immune cells to arrive at sites of tissue injury and are thought to initiate the necessary inflammatory response required to restore tissue integrity (36). We therefore next sought to determine whether PR1P could be used to reduce neutrophil migration into injured lungs and/or GI tract following tissue injury in established animal models. Lung injury models were chosen to characterize the effect of PR1P on inflammation from diverse insults and injury mechanisms, and over different time courses. Specifically, lung injury was induced either by inhalation of lipopolysaccharide (LPS, a component of the bacterial cell wall and used as a potent trigger of acute lung inflammation whereby neutrophils migrate into the lungs within 4-24 hours (37)) or by intra-tracheal administration of bleomycin (38) (which induces acute inflammation and later fibrosis through a multi-factorial mechanism), or HCL (39) (also multifactorial in nature through damage to the alveolar-capillary membrane (40) resulting in immediate polymorphonuclear neutrophil (PMN) recruitment (41), pulmonary edema, and hypoxia (42)). In each of the lung injury models, tissue injury was followed by daily inhalation of PR1P or scrambled peptide (SP) until completion of each of the experiments according to the injury model (i.e. LPS and HCL for 24 hours, and bleomycin for 72 hours). FACS analyses of BALF following HCL- and bleomycin-induced lung injury revealed that inhaled PR1P significantly reduced toxin-induced neutrophil migration into the airways (i.e. BALF, HCl, FIG. 4A, Bleomycin, FIG. 4C). Representative Diff-Quik@ stained cells used in the FACS analyses following acid injury are shown in FIG. 4B. Similarly, FACS analyses of cells from whole lung digests from LPS injured lungs show that inhaled PR1P significantly reduced the presence of migrated neutrophils into the lungs 24 h after exposure to LPS (FIGS. 4D-4E).

To determine whether reduced neutrophil migration into lungs correlates with reduced inflammation, we measured the levels of pro-inflammatory cytokines in BALF following bleomycin injury and found that inhaled PR1P significantly reduced levels of TNF-α, IL-1β, and, IL-6 compared to scrambled peptide (see FIG. 4F). Furthermore, inhaled PR1P reduced myeloperoxidase (MPO) activity in lung tissue following bleomycin injury (FIG. 4F). Together these data suggest that inhaled PR1P effectively reduces neutrophil migration into the lungs, and in so doing PR1P attenuates lung inflammation as determined by reduced cytokine levels and MPO activity.

PR1P improves disease parameters in TNBS-induced colitis in

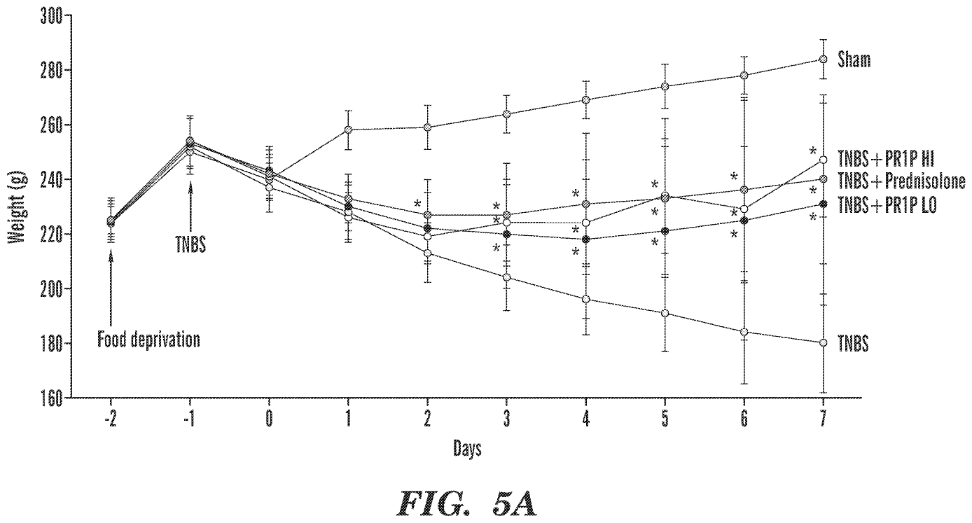

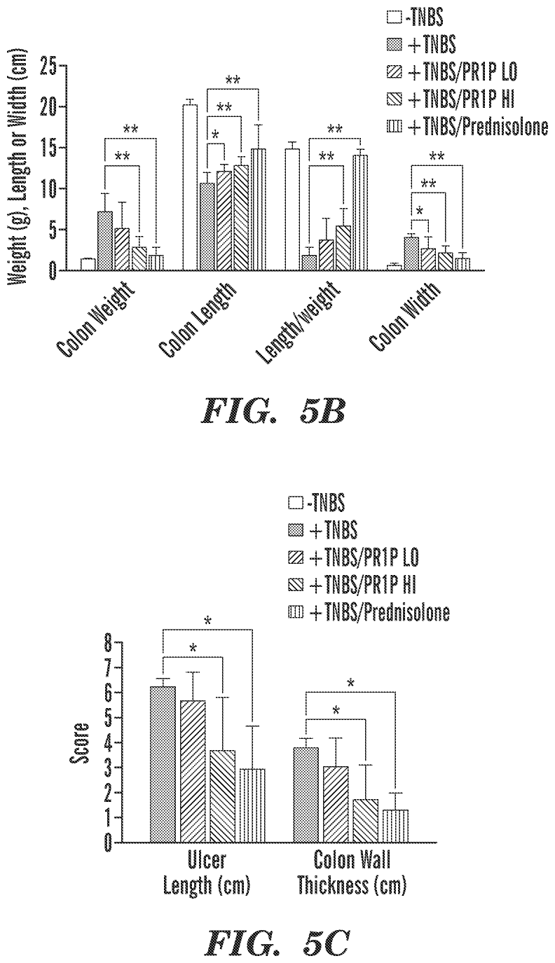

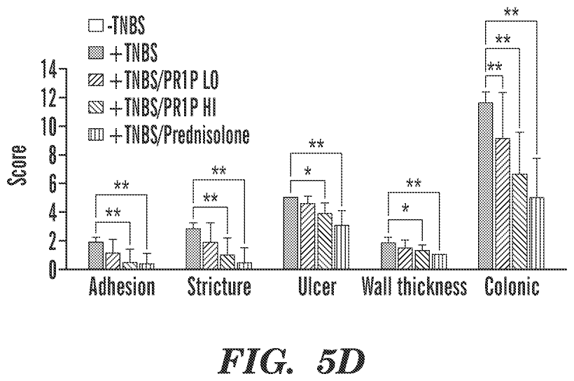

We next characterized the anti-inflammatory and potential therapeutic activity of PR1P in a TNBS-induced ulcerative colitis model (33). In this model, animals treated with TNBS develop acute colitis with bloody, mucus laden diarrhea and sustained weight loss that lasts UP TO three weeks (33). Induction of TNBS colitis (on Day 0) resulted in a decrease in body weight in all experimental groups (FIG. 5A). Treatment with PR1P (low and high doses) and with prednisolone (which served as a positive control) significantly improved weights as early as Day 3 (PR1P, both doses) and Day 2 (Prednisolone) following injury as indicated. Upon study completion on Day 7, the colons of each animal were removed and measured for length and weight and examined macroscopically for ulcers. Both PR1P (high dose) and Prednisolone significantly attenuated TNBS-induced gain in colon weight (FIG. 5B) and both low and high dose PR1P, and Prednisolone attenuated TNBS-induced colon shortening and widening (FIG. 5B). Also, high dose PR1P and prednisolone each significantly reduced the TNBS-induced drop in the Length/Weight index (FIG. 5B). Macroscopic examination of the colons also revealed that high-dose PR1P and prednisolone significantly attenuated increases in TNBS-induced ulcer length and colon wall thickness (FIG. 5C) (Note the grades of Ulcer Lengths and Colon Wall Thickness in the control groups (i.e.-TNBS) are not visible in the graphs because their values are 0). Further macroscopic analysis and grading of the colons revealed that both high-dose PR1P and prednisolone significantly reduced TNBS-induced effects on adhesions, strictures, ulcers, and colon wall thickness (FIG. 5D, note that each condition is graded 0-2, see Methods). Importantly, both low- and high-dose PR1P, and prednisolone significantly reduced the overall Colonic Score (Sums of the above conditions, Maximum score of 12, see Methods). These results suggest that PR1P significantly reduces critical disease indices following exposure to TNBS in rats.

PR1P reduces inflammation in TNBS-induced rat colitis

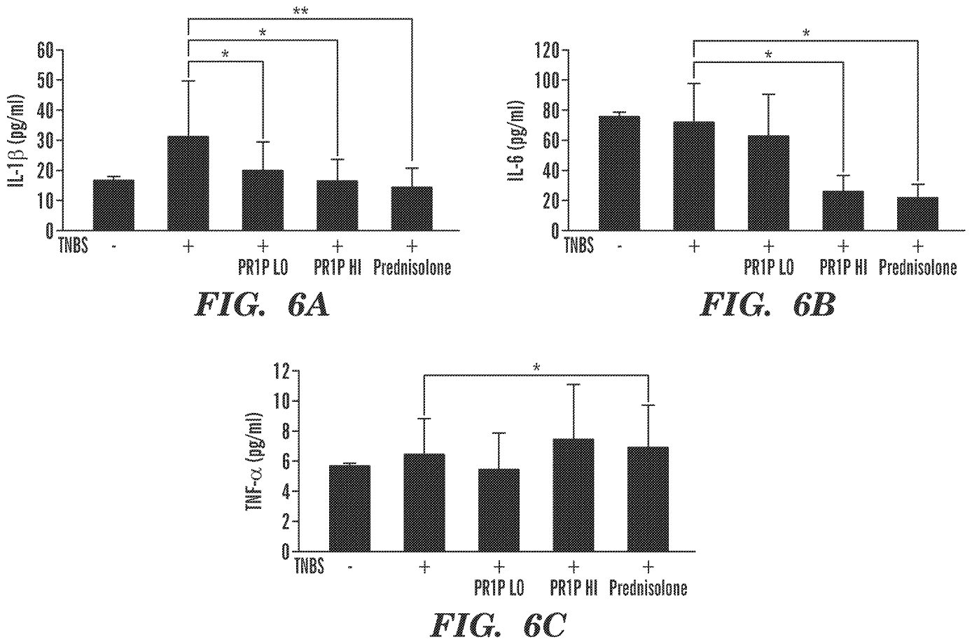

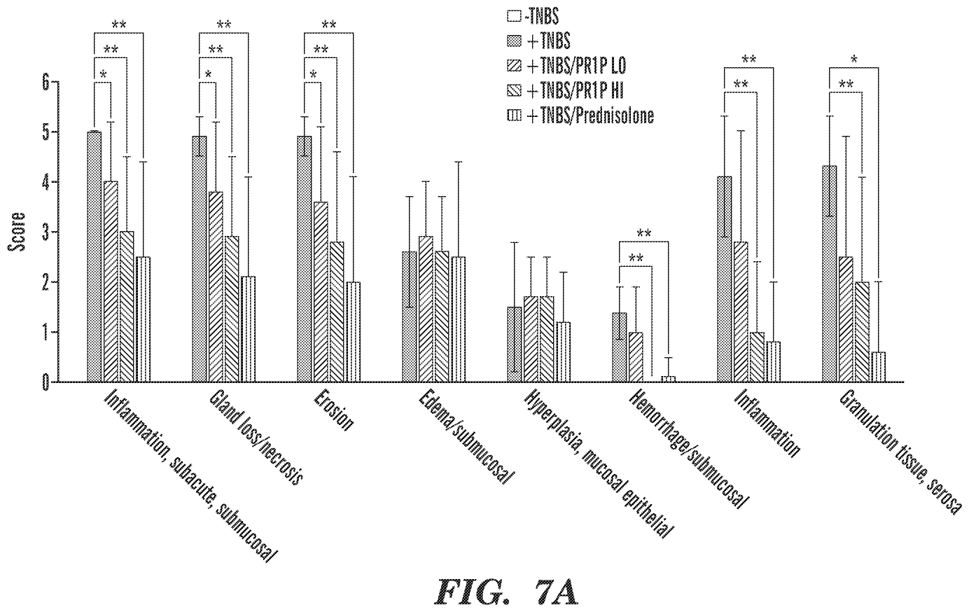

To characterize the anti-inflammatory therapeutic potential of PR1P in TNBS-induced colitis, we quantified plasma levels of the pro-inflammatory mediators IL-1β, IL-6 and TNF-α on Day 7 following necropsy. As shown in FIGS. 6A-6C, PR1P and prednisolone significantly reduced the TNBS-induced increases in plasma IL-1β and IL-6. Interestingly, high-dose PR1P treatment of TNBS-exposed animals led to a significant increase in plasma TNF-α (see Discussion). Together these results suggest that PR1P mediates the serum levels of critical pro-inflammatory cytokines during disease progression in TNBS-induced colitis in rats.

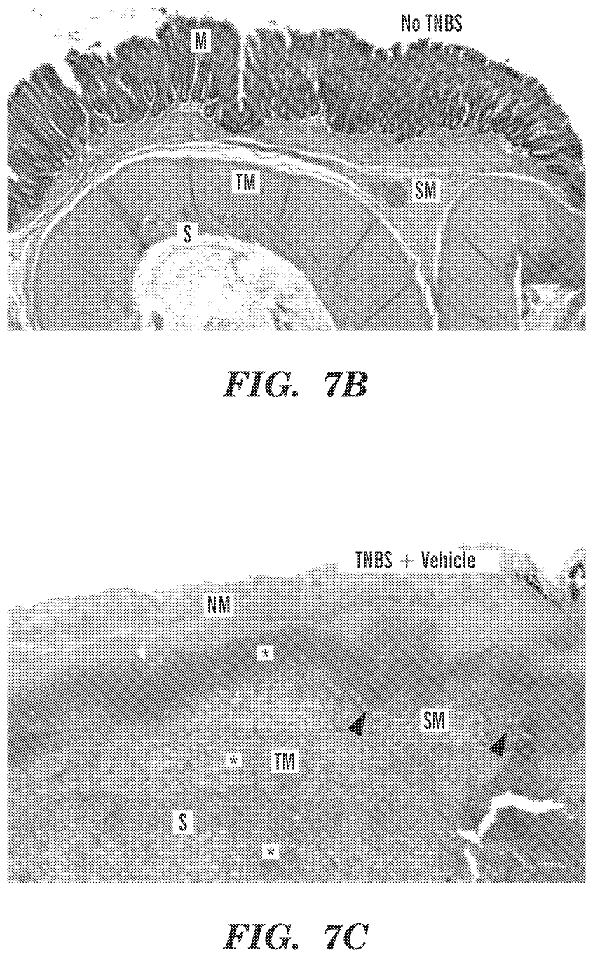

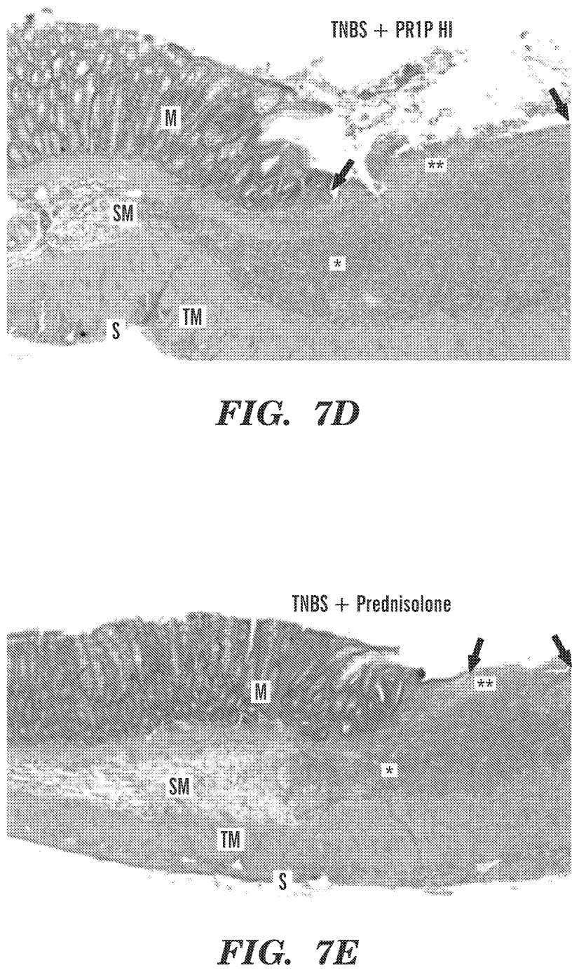

Histopathologic findings in this study were consistent with the TNBS-induced rat colitis model (33), and administration of TNBS was associated with the highest mean histopathology scores for most features, including sub/mucosal inflammation, necrosis, erosion, hemorrhage, transmural inflammation, and serosal granulation tissue (FIG. 7A). As shown in FIG. 7A, both the low- and high dose PR1P, and prednisolone, significantly reduced the effects of TNBS in our model on many features scored including inflammation, gland loss/necrosis, erosion/ulceration, sub-mucosal hemorrhage, and granulation tissue. Representative images of H&E stained colon sections (Day 7) from naïve animals and those exposed to TNBS and indicated treatment protocols are shown in FIGS. 7B-7E and illustrate the effect of PR1P on disease development. It is known that neutrophil migration into the colon begins immediately after injury, peaks at 24 hours, and drops to approximately 50% and 20% of maximum by days 3 and 10, respectively (43). Representative H&E stained images of colonic tissue sections from animals on Day 7 are shown in FIGS. 7B-7E and illustrate the changes reported in FIG. 7A showing PR1P dose-associated reductions in histopathologic features that are similar to prednisolone treated animals.

### DISCUSSION

Here we show that VEGF degradation products generated via VEGF proteolysis by neutrophil elastase are chemoattractant to neutrophils. We subsequently characterized the ability of a novel VEGF binding and stabilizing peptide we developed to mediate endogenous VEGF signaling and inflammation in three murine models of acute lung injury and in a rat ulcerative colitis model in which tissue injuries were induced by diverse mechanisms. Importantly, by binding VEGF within the VEGF heparin-binding domain where proteases are known to bind VEGF (44, 45), PR1P prevents VEGF degradation (46). As we hypothesized, PR1P reduced neutrophil migration into injured tissue and reduced local inflammation following tissue injury. Our results underscore the importance of VEGF in mediating inflammation following tissue injury, and support its importance in maintaining and restoring tissue to health. Furthermore, these studies support a potential role for PR1P in treating diseases commonly caused by tissue injury and characterized by inflammation and VEGF dysregulation.

We chose ARDS and UC animal models to characterize the ability of PR1P to improve outcome because both these diseases are induced by tissue injury and are characterized by local inflammation and VEGF dysregulation (2, 15, 19). Despite widely disparate injury mechanisms, all four models we tested (LPS, acid, bleomycin and TNBS) are known to result in early neutrophil migration into injured tissue and acute inflammation (47-49). Neutrophils are central to innate immunity (50), and are implicated in the onset and progression of many inflammatory diseases, including ARDS (24) and UC (9, 51). Neutrophils are often the first immune cells to arrive at sites of tissue injury and mediate both the initiation and resolution of the inflammatory response that is crucial in returning injured tissue to health (36). Production and release of pro-inflammatory cytokines including tumor necrosis factor (TNF)-α, interleukin (IL)-1β and interleukin (IL)-6 (52), secreted by a variety of immune cells including neutrophils, macrophages, T- and B-cells, dendritic cells, and vascular endothelial cells (53) also follow tissue injury, and together with the appearance of neutrophils in both ARDS and IBD, are central to the initiation and maintenance of inflammation (53). VEGF dysregulation following tissue injury is due to the release by inflammatory cells, including neutrophils, of serine proteases including elastase and plasmin (54, 55) which degrade VEGF into smaller VEGF isomers (VEGF degradation products, fVEGF) with altered VEGF receptor binding and signaling properties (20, 21). Thus, it is not surprising that injury to most tissues, irrespective of mechanism, results in acute inflammation and VEGF dysregulation.

Although it is well documented that VEGF levels and VEGF signaling are dynamic during both ARDS and IBD: it is not clear how this important growth and angiogenic factor mediates disease outcome during illness (15, 18, 19). ARDS is an acute inflammatory disease resulting from diverse direct and indirect insults to the lungs (1) and is characterized by fulminant acute respiratory failure requiring mechanical ventilation and prolonged ICU admission (3). There are no medicines to cure ARDS, and mortality remains high (56). VEGF is widely thought to play a protective role during ARDS progression as VEGF has been shown to mediate the survival of lung airway epithelial cells, and the repair of damaged alveolar capillary membrane barriers thereby reducing pulmonary edema (17, 18). Decreased VEGF levels in the lungs as measured in broncho-alveolar lavage fluid and in exhaled breath condensate (57) of critically ill patients with ARDS are associated with poor prognosis (58, 59). The decline in VEGF levels in the lungs in patients with ARDS is thought to be due to degradation of VEGF by inflammatory cell derived proteases in the alveoli (20, 21). Interestingly, serum concentrations of VEGF increase early in ARDS and return to normal if patients recover (60, 61). This dynamic is thought to be due to release of VEGF from the lungs into the systemic circulation due to impaired epithelial and endothelial barriers (61).

IBD occurs in two forms, ulcerative colitis (UC) and Crohn's disease, which are generally, but not always, distinguished by the portion of the gastrointestinal tract affected by disease (62). IBD is thought to be a consequence of inappropriate and unrelenting activation of the mucosal immune system (63) whereby inflammatory cells including neutrophils, secrete proteases which impair mucosal repair through inhibition of epithelial cell proliferation and through degradation of adhesion and signaling molecules including adherens junction protein, E-cadherin, elastin, collagen, and MMP inhibitors that participate in the maintenance of the intestinal barrier (25, 64-68). In a similar fashion to ARDS, systemic levels of VEGF may elevate during disease due to the release of VEGF from the GI tract from vascular endothelial breakdown (69). Chronic inflammation in IBD is due in part to ongoing production of cytokines by inflammatory cells. There are several drugs available to treat IBD that each target the inflammatory process via different mechanisms and include amino-salicylates, corticosteroids, immunomodulators, and anti-TNF-α antibodies. Despite these multiple medical options, proctocolectomy to remove irreversibly diseased intestine is performed in 30% of patients with IBD (9, 70, 71). Thus, the lack of a medical therapy to treat ARDS, and the fact that one-third of patients with IBD do not respond to therapy (71) highlight the need for the development of an alternative therapeutic such as PR1P to treat these common and debilitating diseases that are characterized by inflammation and VEGF dysregulation.

The mechanism of action of PR1P is through its binding to VEGF within the VEGF Heparin Binding Domain (HBD) thereby impeding the binding of VEGF by proteolytic enzymes. In so doing, PR1P prevents degradation of VEGF into fVEGF, which we demonstrate is a neutrophil chemoattractant, and which therefore stimulates further inflammation. It is intriguing that PR1P had opposite effects on TNF-α levels in the lung and GI tract, respectively, at the different timepoints sampled in our models. Cytokines play an important role in dysregulated immune responses and an imbalance between pro- and anti-inflammatory cytokines can have deleterious effects on disease outcome. Patterns of pro- and anti-inflammatory cytokine levels are dynamic over the course of specific diseases and cytokine levels reflect animal- and organ-specific stages of illness. Also, TNF-α was recently shown to have anti-inflammatory properties in a dextran sodium sulfate (DSS)-induced colitis model (72). Regardless of mechanism, results of these studies support our own and point to a critical role for PR1P in mediating inflammation. VEGF therapy involves strategies to deliver VEGF (through direct delivery of VEGF or modified VEGF), or via gene therapy to increase local tissue VEGF concentrations (73). Although successful in animal models, VEGF therapy has proven unsuccessful in human studies for many reasons, including that the treatment of acute or chronically inflamed tissue with VEGF likely leads to rapid VEGF degradation and increased local concentrations of fVEGF due to elevated concentration of proteases. This therapeutic approach fails to upregulate VEGF signaling locally and instead likely stimulates further inflammation. Thus, PR1P is fundamentally different than conventional VEGF therapy as PR1P stabilizes endogenous VEGF within local tissue microenvironments, preserves endogenous VEGF signaling, and reduces the production of fVEGF.

As noted, PR1P binds VEGF within the VEGF HBD (46) thereby competing with proteases for binding to VEGF. The HBD is an important carboxy-terminal region on VEGF that plays a critical role in VEGF signaling and therefore may serve as an important target for drug development. Disparities in HBD expression between alternate splice forms of different VEGF isoforms result in subtle variations in downstream signaling (74, 75). For example, proteolytic degradation of the whole or parts of the carboxy-terminal region of the VEGF HBD significantly reduced VEGF-induced mitosis in human vascular endothelial cells (22, 76). Furthermore, Kurtagic et al. showed that neutrophil-derived elastase (NE) cleaved VEGF within internal regions of the N-terminus of VEGF in addition to regions at the C-terminus (20, 21). They subsequently showed that NE generated VEGF fragments (fVEGF) demonstrated higher binding affinity to VEGF Receptor 1 (VEGFR1) compared to VEGFR2 (20, 21) which is significant because VEGFR1 is expressed on neutrophils (77). Thus, in addition to attracting neutrophils into sites of tissue injury, fVEGF also likely upregulates neutrophil activity once present within the inflamed tissue. Collectively, these findings provide key evidence that the VEGF HBD may play a central role in mediating VEGF signaling during both health and in disease. Keyt et al. showed that chronic wounds are associated with a high concentration of proteases, elevated levels of fVEGF, and decreased VEGF activity (44). Treatment of chronic wounds with a mutant VEGF containing a modified HBD that prevents plasmin binding to VEGF resulted in increased VEGF stability within the wound that resulted in increased angiogenesis (in surrounding wound tissue) and improved wound healing (44). It is well known that VEGF signaling controls a wide variety of biological and functional outcomes (78, 79) that are important in maintenance of tissue health and healing (12, 80). Although the signaling and binding properties of fVEGF are not fully elucidated, our data and those of others suggest that fVEGF may serve as a point of convergence in VEGF signaling that may determine whether tissues begin to heal and remodel or remain inflamed.

In summary, we investigated the potential link between neutrophils, often the first immune cell to arrive at the site of tissue injury from multiple disparate direct and indirect insults, and VEGF, a ubiquitous signaling molecule implicated in mediating healing of injured tissues. We discovered that fVEGF, a product of VEGF degradation by inflammatory cell derived proteases, is chemoattractant to neutrophils. Furthermore, we showed that PR1P, a VEGF binding and stabilizing peptide reduced neutrophil migration into injured tissue and inflammation in animal models of ARDS and UC. PR1P is a distinct alternative to conventional VEGF therapy. Our studies underscore the importance of VEGF in mediating tissue during both health and disease and also point to fVEGF serving as a point of convergence in VEGF signaling that may determine whether tissues begin to heal and remodel or remain inflamed. To date there has been little work done in developing pharmaceuticals for ARDS and UC that target VEGF. Our work suggests that drug development to support PR1P or other molecules targeting fVEGF biology may prove impactful in treating diseases like ARDS or UC that are characterized by VEGF dysregulation and inflammation.

### REFERENCE

1. Matthay M A, Zemans R L, Zimmerman G A, Arabi Y M, Beitler J R, Mercat A, et al., Acute respiratory distress syndrome. Nat Rev Dis Primers (2019) 5:18. doi: 10.1038/s41572-019-0069-0
2. Zdravkovic N D, Jovanovic I P, Radosavljevic G D, Arsenijevic A N, Zdravkovic N D, Mitrovic S, et al., Potential dual immunomodulatory role of VEGF in ulcerative colitis and colorectal carcinoma. Int J Med Sci (2014) 11:936-47. doi: 10.7150/ijms.8277
3. Banavasi H, Nguyen P, Osman H, Soubani A O. Management of ARDS-what works and what does not. Am J Med Sci (2021) 362:13-23. doi: 10.1016/j.amjms.2020.12.019
4. Lynch W D, Hsu R. StatPearls. Treasure Island, Florida, USA: StatPearls Publishing LLC; (2022). [Google Scholar]
5. Ferretti F, Cannatelli R, Monico M C, Maconi G, Ardizzone S. An update on current pharmacotherapeutic options for the treatment of ulcerative colitis. J Clin Med (2022) 11. doi: 10.3390/jcm11092302
6. Liew P X, Kubes P. The neutrophil's role during health and disease. Physiol Rev (2019) 99:1223-48. doi: 10.1152/physrev.00012.2018
7. Beeh K M, Kornmann O, Buhl R, Culpitt S V, Giembycz M A, Barnes P J, et al., Neutrophil chemotactic activity of sputum from patients with COPD: role of interleukin 8 and leukotriene B4. Chest (2003) 123:1240-7. doi: 10.1378/chest. 123.4.1240
8. Moldoveanu B, Otmishi P, Jani P, Walker J, Sarmiento X, Guardiola J, et al., Inflammatory mechanisms in the lung. J Inflammation Res (2009) 2:1-11.
9. Podolsky D K. Inflammatory bowel disease. N Engl J Med (2002) 347:417-29. doi: 10.1056/NEJMra020831
10. Wera O, Lancellotti P, Oury C. The dual role of neutrophils in inflammatory bowel diseases. J Clin Med (2016) 5. doi: 10.3390/jcm5120118
11. Frigerio S, Lartey D A, D′Haens G R, Grootjans J. The role of the immune system in IBD-associated colorectal cancer: from pro to anti-tumorigenic mechanisms. Int J Mol Sci (2021) 22. doi: 10.3390/ijms222312739
12. Ferrara N. Vascular endothelial growth factor: basic science and clinical progress. Endocr Rev (2004) 25:581-611. doi: 10.1210/er.2003-0027
13. Apte R S, Chen D S, Ferrara N. VEGF in signaling and disease: beyond discovery and development. Cell (2019) 176:1248-64. doi: 10.1016/j.cell.2019.01.021
14. Ferrara N, Gerber H P, LeCouter J. The biology of VEGF and its receptors. Nat Med (2003) 9:669-76. doi: 10.1038/nm0603-669
15. Barratt S, Medford A R, Millar A B. Vascular endothelial growth factor in acute lung injury and acute respiratory distress syndrome. Respiration (2014) 87:329-42. doi: 10.1159/000356034
16. Holmes D I, Zachary I. The vascular endothelial growth factor (VEGF) family: angiogenic factors in health and disease. Genome Biol (2005) 6:209. doi: 10.1186/gb-2005-6-2-209
17. Medford A R, Millar A B. Vascular endothelial growth factor (VEGF) in acute lung injury (ALI) and acute respiratory distress syndrome (ARDS): paradox or paradigm? Thorax (2006) 61:621-6. doi: 10.1136/thx.2005.040204
18. Medford A R L, Godinho S I H, Keen L J, Bidwell J L, Millar A B. Relationship between vascular endothelial growth factor+936 genotype and plasma/epithelial lining fluid vascular endothelial growth factor protein levels in patients with and at risk for ARDS. Chest (2009) 136:457-64. doi: 10.1378/chest.09-0383
19. Mateescu R B, Bastian A E, Nichita L, Marinescu M, Rouhani F, Voiosu A M, et al., Vascular endothelial growth factor-key mediator of angiogenesis and promising therapeutical target in ulcerative colitis. Rom J Morphol Embryol (2017) 58:1339-45. [PubMed] [Google Scholar]
20. Kurtagic E, Jedrychowski M P, Nugent M A. Neutrophil elastase cleaves VEGF to generate a VEGF fragment with altered activity. Am J Physiol Lung Cell Mol Physiol (2009) 296: L534-546. doi: 10.1152/ajplung.90505.2008
21. Kurtagic E, Rich C B, Buczek-Thomas J A, Nugent M A. Neutrophil elastase-generated fragment of vascular endothelial growth factor-a stimulates macrophage and endothelial progenitor cell migration. PloS One (2015) 10: e0145115. doi: 10.1371/journal.pone.0145115
22. Lauer G, Sollberg S, Cole M, Flamme I, Sturzebecher J, Mann K, et al., Expression and proteolysis of vascular endothelial growth factor is increased in chronic wounds. J Invest Dermatol (2000) 115:12-8. doi: 10.1046/j.1523-1747.2000.00036.x
23. Kawabata K, Hagio T, Matsuoka S. The role of neutrophil elastase in acute lung injury. Eur J Pharmacol (2002) 451:1-10. doi: 10.1016/s0014-2999 (02) 02182-9
24. Grommes J, Soehnlein O. Contribution of neutrophils to acute lung injury. Mol Med (2011) 17:293-307. doi: 10.2119/molmed.2010.00138
25. Kuno Y, Ina K, Nishiwaki T, Tsuzuki T, Shimada M, Imada A, et al., Possible involvement of neutrophil elastase in impaired mucosal repair in patients with ulcerative colitis. J Gastroenterol (2002) 37 Suppl 14:22-32. doi: 10.1007/BF03326409
26. Adini A, Adini I, Chi Z L, Derda R, Birsner A E, Matthews B D, et al., A novel strategy to enhance angiogenesis in vivo using the small VEGF-binding peptide PR1P. Angiogenesis (2017) 20:399-408. doi: 10.1007/s10456-017-9556-7
27. Adini A, Wu H, Dao D T, Ko V H, Yu L J, Pan A, et al., PR1P stabilizes VEGF and upregulates its signaling to reduce elastase induced murine emphysema. Am J Respir Cell Mol Biol (2020) 63:452-63. doi: 10.1165/rcmb.2019-04340C
28. Adini A, Adini I, Grad E, Tal Y, Danenberg H D, Kang P M, et al., The prominin-1-Derived peptide improves cardiac function following ischemia. Int J Mol Sci (2021) 22. doi: 10.3390/ijms22105169
29. Chi Z L, Adini A, Birsner A E, Bazinet L, Akula J D, D'Amato R J, et al., PR1P ameliorates neurodegeneration through activation of VEGF signaling pathway and remodeling of the extracellular environment. Neuropharmacology (2019) 148:96-106. doi: 10.1016/j.neuropharm.2018.12.029
30. Yin J, Shi C, He W, Yan W, Deng J, Zhang B, et al., Specific bio-functional CBD-PR1P peptide binding VEGF to collagen hydrogels promotes the recovery of cerebral ischemia in rats. J BioMed Mater Res A (2022) 110:1579-89. doi: 10.1002/jbm.a.37409
31. Yuan Z, Sheng D, Jiang L, Shafiq M, Khan A U R, Hashim R, et al., Vascular endothelial growth factor-capturing aligned electrospun Polycaprolactone/Gelatin nanofibers promote patellar ligament regeneration. Acta Biomater (2022) 140:233-46. doi: 10.1016/j.actbio.2021.11.040
32. Chen Y, Yuan Z, Sun W, Shafiq M, Zhu J, Chen J, et al., Vascular endothelial growth factor-recruiting nanofiber bandages promote multifunctional skin regeneration via improved angiogenesis and immunomodulation. Advanced Fiber Materials (2023) 5:327-48. doi: 10.1007/s42765-022-00226-8
33. Antoniou E, Margonis G A, Angelou A, Pikouli A, Argiri P, Karavokyros I, et al., The TNBS-induced colitis animal model: an overview. Ann Med Surg (Lond) (2016) 11:9-15. doi: 10.1016/j.amsu.2016.07.019
34. Kawada M, Arihiro A, Mizoguchi E. Insights from advances in research of chemically induced experimental models of human inflammatory bowel disease. World J Gastroenterol (2007) 13:5581-93. doi: 10.3748/wjg.v13.i42.5581
35. Morris G P, Beck P L, Herridge M S, Depew W T, Szewczuk M R, Wallace J L, et al., Hapten-induced model of chronic inflammation and ulceration in the rat colon. Gastroenterology (1989) 96:795-803. doi: 10.1016/S0016-5085 (89) 80079-4
36. Selders G S, Fetz A E, Radic M Z, Bowlin G L. An overview of the role of neutrophils in innate immunity, inflammation and host-biomaterial integration. Regener Biomater (2017) 4:55-68. doi: 10.1093/rb/rbw041
37. Chen H, Bai C, Wang X. The value of the lipopolysaccharide-induced acute lung injury model in respiratory medicine. Expert Rev Respir Med (2010) 4:773-83. doi: 10.1586/ers.10.71
38. Ge V, Banakh I, Tiruvoipati R, Haji K. Bleomycin-induced pulmonary toxicity and treatment with infliximab: a case report. Clin Case Rep (2018) 6:2011-4. doi: 10.1002/ccr3.1790
39. Zarbock A, Singbartl K, Ley K. Complete reversal of acid-induced acute lung injury by blocking of platelet-neutrophil aggregation. J Clin Invest (2006) 116:3211-9. doi: 10.1172/JCI29499
40. Eijking E P, Gommers D, So K L, Vergeer M, Lachmann B. Surfactant treatment of respiratory failure induced by hydrochloric acid aspiration in rats. Anesthesiology (1993) 78:1145-51. doi: 10.1097/00000542-199306000-00019
41. Goldman G, Welbourn R, Kobzik L, Valeri C R, Shepro D, Hechtman H B, et al., Reactive oxygen species and elastase mediate lung permeability after acid aspiration. J Appl Physiol (1985) (1992) 73:571-5. doi: 10.1152/jappl.1992.73.2.571
42. Nagase T, Uozumi N, Ishii S, Kume K, Izumi T, Ouchi Y, et al., Acute lung injury by sepsis and acid aspiration: a key role for cytosolic phospholipase A2. Nat Immunol (2000) 1:42-6. doi: 10.1038/76897
43. van Lierop P P, de Haar C, Lindenbergh-Kortleve D J, Simons-Oosterhuis Y, van Rijt L S, Lambrecht B N, et al., T-Cell regulation of neutrophil infiltrate at the early stages of a murine colitis model. Inflammation Bowel Dis (2010) 16:442-51. doi: 10.1002/ibd.21073
44. Keyt B A, Berleau L T, Nguyen H V, Chen H, Heinsohn H, Vandlen R, et al., The carboxyl-terminal domain (111-165) of vascular endothelial growth factor is critical for its mitogenic potency. J Biol Chem (1996) 271:7788-95. doi: 10.1074/jbc.271.13.7788
45. Roth D, Piekarek M, Paulsson M, Christ H, Bloch W, Krieg T, et al., Plasmin modulates vascular endothelial growth factor-a-mediated angiogenesis during wound repair. Am J Pathol (2006) 168:670-84. doi: 10.2353/ajpath.2006.050372
46. Adini A, Wu H, Dao D T, Ko V H, Yu LJ, Pan A, et al., PR1P stabilizes VEGF and upregulates its signaling to reduce elastase-induced murine emphysema. Am J Respir Cell Mol Biol (2020) 63:452-63. doi: 10.1165/rcmb.2019-04340C
47. Wilson M S, Wynn T A. Pulmonary fibrosis: pathogenesis, etiology and regulation. Mucosal Immunol (2009) 2:103-21. doi: 10.1038/mi.2008.85
48. Fournier B M, Parkos C A. The role of neutrophils during intestinal inflammation. Mucosal Immunol (2012) 5:354-66. doi: 10.1038/mi.2012.24
49. Van de Louw A, Jean D, Frisdal E, Cerf C, d′Ortho M P, Baker A H, et al., Neutrophil proteinases in hydrochloric acid- and endotoxin-induced acute lung injury: evaluation of interstitial protease activity by in situ zymography. Lab Invest (2002) 82:133-45. doi: 10.1038/labinvest.3780406
50. Kraus R F, Gruber M A. Neutrophils-from bone marrow to first-line defense of the innate immune system. Front Immunol (2021) 12:767175. doi: 10.3389/fimmu.2021.767175
51. Camba-Gomez M, Arosa L, Gualillo O, Conde-Aranda J. Chemokines and chemokine receptors in inflammatory bowel disease: recent findings and future perspectives. Drug Discovery Today (2022) 27:1167-75. doi: 10.1016/j.drudis.2021.12.004
52. Donnelly S C, MacGregor I, Zamani A, Gordon M W, Robertson C E, Steedman D J, et al., Plasma elastase levels and the development of the adult respiratory distress syndrome. Am J Respir Crit Care Med (1995) 151:1428-33. doi: 10.1164/ajrccm. 151.5.7735596
53. Kany S, Vollrath J T, Relja B. Cytokines in inflammatory disease. Int J Mol Sci (2019) 20. doi: 10.3390/ijms20236008
54. Bank U, Ansorge S. More than destructive: neutrophil-derived serine proteases in cytokine bioactivity control. J Leukoc Biol (2001) 69:197-206. doi: 10.1189/jlb.69.2.197
55. Lee K H, Lee C H, Jeong J, Jang A H, Yoo C G. Neutrophil elastase differentially regulates interleukin 8 (IL-8) and vascular endothelial growth factor (VEGF) production by cigarette smoke extract. J Biol Chem (2015) 290:28438-45. doi: 10.1074/jbc.M115.663567
56. Nanchal R S, Truwit J D. Recent advances in understanding and treating acute respiratory distress syndrome. F1000Res (2018) 7. doi: 10.12688/f1000research. 15493.1
57. Chen J, Lv X, He H, Qi F, Chen J. Significance of vascular endothelium growth factor testing in exhaled breath condensate of patients with acute respiratory distress syndrome. Technol Health Care (2020) 28:347-54. doi: 10.3233/THC-209035
58. Abadie Y, Bregeon F, Papazian L, Lange F, Chailley-Heu B, Thomas P, et al., Decreased VEGF concentration in lung tissue and vascular injury during ARDS. Eur Respir J (2005) 25:139-46. doi: 10.1183/09031936.04.00065504
59. Maitre B, Boussat S, Jean D, Gouge M, Brochard L, Housset B, et al., Vascular endothelial growth factor synthesis in the acute phase of experimental and clinical lung injury. Eur Respir J (2001) 18:100-6. doi: 10.1183/09031936.01.00074701
60. Tomita K, Saito Y, Suzuki T, Imbaby S, Hattori K, Matsuda N, et al., Vascular endothelial growth factor contributes to lung vascular hyperpermeability in sepsis-associated acute lung injury. Naunyn Schmiedebergs Arch Pharmacol (2020) 393:2365-74. doi: 10.1007/s00210-020-01947-6
61. Thickett D R, Armstrong L, Christie S J, Millar A B. Vascular endothelial growth factor may contribute to increased vascular permeability in acute respiratory distress syndrome. Am J Respir Crit Care Med (2001) 164:1601-5. doi: 10.1164/ajrccm.164.9.2011071
62. McDowell C, Farooq U, Haseeb M. StatPearls. Treasure Island, Florida, USA: StatPearls Publishing LLC; (2022).
63. Keighley M R, Stockbrugger R W. Inflammatory bowel disease. Aliment Pharmacol Ther (2003) 18 Suppl 3:66-70. doi: 10.1046/j.0953-0673.2003.01727.x
64. Ginzberg H H, Cherapanov V, Dong Q, Cantin A, McCulloch C A, Shannon P T, et al., Neutrophil-mediated epithelial injury during transmigration: role of elastase. Am J Physiol Gastrointest Liver Physiol (2001) 281: G705-717. doi: 10.1152/ajpgi.2001.281.3.G705
65. Ginzberg H H, Shannon P T, Suzuki T, Hong O, Vachon E, Moraes T, et al., Leukocyte elastase induces epithelial apoptosis: role of mitochondial permeability changes and akt. Am J Physiol Gastrointest Liver Physiol (2004) 287: G286-298. doi: 10.1152/ajpgi.00350.2003
66. Morohoshi Y, Matsuoka K, Chinen H, Kamada N, Sato T, Hisamatsu T, et al., Inhibition of neutrophil elastase prevents the development of murine dextran sulfate sodium-induced colitis. J Gastroenterol (2006) 41:318-24. doi: 10.1007/s00535-005-1768-8
67. Geraghty P, Rogan M P, Greene C M, Boxio R M, Poiriert T, O'Mahony M, et al., Neutrophil elastase up-regulates cathepsin b and matrix metalloprotease-2 expression. J Immunol (2007) 178:5871-8. doi: 10.4049/jimmunol.178.9.5871
68. Gordon M H, Chauvin A, Boisvert F M, MacNaughton W K. Proteolytic processing of the epithelial adherens junction molecule e-cadherin by neutrophil elastase generates short peptides with novel wound-healing bioactivity. Cell Mol Gastroenterol Hepatol (2019) 7:483-486 e488. doi: 10.1016/j.jcmgh.2018.10.012
69. Staels W, Heremans Y, Heimberg H, De Leu N. VEGF-a and blood vessels: a beta cell perspective. Diabetologia (2019) 62:1961-8. doi: 10.1007/s00125-019-4969-z
70. McLean M H, Neurath M F, Durum S K. Targeting interleukins for the treatment of inflammatory bowel disease-what lies beyond anti-TNF therapy? Inflammation Bowel Dis (2014) 20:389-97. doi: 10.1097/01.MIB.0000437616.37000.41
71. Bernstein C N. Treatment of IBD: where we are and where we are going. Am J Gastroenterol (2015) 110:114-26. doi: 10.1038/ajg.2014.357
72. Naito Y, Takagi T, Handa O, Ishikawa T, Nakagawa S, Yamaguchi T, et al., Enhanced intestinal inflammation induced by dextran sulfate sodium in tumor necrosis factor-alpha deficient mice. J Gastroenterol Hepatol (2003) 18:560-9. doi: 10.1046/j.1440-1746.2003.03034.x
73. Jones M K, Kawanaka H, Baatar D, Szabo I L, Tsugawa K, Pai R, et al., Gene therapy for gastric ulcers with single local injection of naked DNA encoding VEGF and angiopoietin-1. Gastroenterology (2001) 121:1040-7. doi: 10.1053/gast.2001.29308
74. Park J E, Keller G A, Ferrara N. The vascular endothelial growth factor (VEGF) isoforms: differential deposition into the subepithelial extracellular matrix and bioactivity of extracellular matrix-bound VEGF. Mol Biol Cell (1993) 4:1317-26. doi: 10.1091/mbc.4.12.1317
75. Carmeliet P, Ng Y S, Nuyens D, Theilmeier G, Brusselmans K, Cornelissen I, et al., Impaired myocardial angiogenesis and ischemic cardiomyopathy in mice lacking the vascular endothelial growth factor isoforms VEGF164 and VEGF188. Nat Med (1999) 5:495-502. doi: 10.1038/8379
76. Lauer G, Sollberg S, Cole M, Krieg T, Eming S A. Generation of a novel proteolysis resistant vascular endothelial growth factor165 variant by a site-directed mutation at the plasmin sensitive cleavage site. FEBS Lett (2002) 531:309-13. doi: 10.1016/S0014-5793 (02) 03545-7
77. Massena S, Christoffersson G, Vagesjo E, Seignez C, Gustafsson K, Binet F, et al., Identification and characterization of VEGF-a-responsive neutrophils expressing CD49d, VEGFR1, and CXCR4 in mice and humans. Blood (2015) 126:2016-26. doi: 10.1182/blood-2015-03-631572
78. Shibuya M. Structure and function of VEGF/VEGF-receptor system involved in angiogenesis. Cell Struct Funct (2001) 26:25-35. doi: 10.1247/csf.26.25
79. Ferrara N. Vascular endothelial growth factor and the regulation of angiogenesis. Recent Prog Horm Res (2000) 55:15-35; discussion 35-16.
80. Senger D R, Ledbetter S R, Claffey K P, Papadopoulos-Sergiou A, Peruzzi C A, Detmar M. Stimulation of endothelial cell migration by vascular permeability factor/vascular endothelial growth factor through cooperative mechanisms involving the alphavbeta3 integrin, osteopontin, and thrombin. Am J Pathol (1996) 149:293-305.

1. A peptide comprising the sequence of SEQ ID NO: 1 (DRVQRQTTTVVA).

2. The peptide of claim 1, consisting of the sequence of SEQ ID NO: 1.

3. A pharmaceutical composition comprising the peptide of claim 1 and a pharmaceutically acceptable excipient.

4. A method of treating inflammation in a subject in need thereof, the method comprising administering an anti-inflammatory or anti-infective treatment to a subject with an increased level of fragmented VEGF (fVEGF).

5. The method of claim 4, wherein the subject is a subject determined to have an increased level of fragmented VEGF (fVEGF).

6. The method of claim 4, further comprising a first step of detecting or measuring the level of fragmented VEGF (fVEGF) in the subject or a sample obtained from the subject.

7. The method of claim 4, wherein the anti-inflammatory reduces the rate of formation of fVEGF.

8. (canceled)

9. A method of treating inflammation or infection in a subject in need thereof, the method comprising administering the peptide of claim 1 to the subject.

10-29. (canceled)

30. The method of claim 9, wherein the subject has an increased level of fragmented VEGF (fVEGF).

31. The method of claim 30, further comprising a first step of detecting or measuring the level of fragmented VEGF (fVEGF) in the subject or a sample obtained from the subject.

32. The method of 30, wherein the fVEGF is VEGF₁₆₅.

33. The method of claim 30, wherein the level of fVEGF is the level of fVEGF in the blood of the subject, the level of fVEGF in a blood sample of the subject, the level of fVEGF in an injured or infected tissue of the subject, the level of fVEGF in an injured or infected tissue sample of the subject, the local level of fVEGF, the level of fVEGF at a site of injury and/or infection, the level of fVEGFin the blood of the subject, the level of fVEGF in the serum of the subject, the level of fVEGF in the plasma of the subject, the level of fVEGF in the eye humor of the subject, the level of fVEGF in the saliva of the subject, the level of fVEGF in the urine of the subject, the level of fVEGF in the bronchoalveolar lavage fluid of the subject, the level of fVEGF in the cerebrospinal fluid of the subject, the level of fVEGF in the exhaled breath condensate of the subject, the level of fVEGF in a blood sample of the subject, the level of fVEGF in a serum sample of the subject, the level of fVEGF in a plasma sample of the subject, the level of fVEGF in an eye humor sample of the subject, the level of fVEGF in a saliva sample of the subject, the level of fVEGF in a urine sample of the subject, the level of fVEGF in a bronchoalveolar lavage fluid sample of the subject, the level of fVEGF in a cerebrospinal fluid sample of the subject, or the level of fVEGF in a exhaled breath condensate sample of the subject.

34. The method of claim 30, wherein the level of fVEGF is not the systemic level of fVEGF.

35. The method of claim 30, wherein the fVEGF comprises, consists of, or consists essentially of:
(SEQ ID NO: 2)
ARQENPCGPCSERRKHLVQDPQTCKCSCKNTDSRCKARQLELNERTCRC
DKPRR.


SEQUENCE LISTING (13 sequences)

SEQ ID NO: 1 - protein, 12 aa, synthetic construct
DRVQRQTTTVVA

SEQ ID NO: 2 - protein, 54 aa, synthetic construct
ARQENPCGPCSERRKHLVQDPQTCKCSCKNTDSRCKARQLELNERTCRCDKPRR

SEQ ID NO: 3 - protein, 359 aa, synthetic construct
MALVLGSLLLLGLCGNSFSGGQPSSTDAPKAWNYELPATNYETQDSHKAGPIGILFELVHIFLYVVQPRDFPEDTLRKFLQKAYESKIDYDKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIP

SEQ ID NO: 4 - protein, 485 aa, synthetic construct
GIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNMENGNNGYHKDHVYGIHNPVMTSPSQH

SEQ ID NO: 5 - protein, 368 aa, synthetic construct
MALVLGSLLLLGLCGNSFSGGQPSSTDAPKAWNYELPATNYETQDSHKAGPIGILFELVHIFLYVVQPRDFPEDTLRKFLQKAYESKIDYDKPETVILGLKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIP

SEQ ID NO: 6 - protein, 462 aa, synthetic construct
GIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNPSQH

SEQ ID NO: 7 - protein, 454 aa, synthetic construct
GIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDPSQH

SEQ ID NO: 8 - protein, 462 aa, synthetic construct; identical to SEQ ID NO: 6

SEQ ID NO: 9 - skipped

SEQ ID NO: 10 - protein, 454 aa, synthetic construct; identical to SEQ ID NO: 7

SEQ ID NO: 11 - protein, 485 aa, synthetic construct; identical to SEQ ID NO: 4

SEQ ID NO: 12 - protein, 53 aa, synthetic construct
ARQENPCGPCSERRKHLVQDPQTCKCSCKNTDSRCKARQLELNERTCRCDKPR

SEQ ID NO: 13 - protein, 12 aa, synthetic construct
VRQVVTARDTTQ